(19) United States
(12) Patent Application Publication
(10) Pub. No.: US 2026/0345896 A1
(43) Pub. Date: Nov. 5, 2026

(54) DRUG DELIVERY DEVICE

(71) Applicant: JANSSEN BIOTECH, INC., HORSHAM, PA (US)

(72) Inventor: CURT BINNER, Furlong, PA (US)

(21) Appl. No.: 19/487,816

(22) PCT Filed: Jun. 4, 2024
(86) PCT No.: PCT/IB2024/055453

§ 371 Date: Nov. 25, 2025

Related U.S. Application Data
(60) Provisional application No. 63/508,591, filed on Jun. 16, 2023.
(60) Provisional application No. 63/532,275, filed on Aug. 11, 2023.

(51) Int. Cl. A61M 5/145; A61M 5/142; A61M 5/315

(52) U.S. Cl. CPC A61M 5/14566; A61M 5/14248; A61M 5/31578; A61M 2005/14252; A61M 2005/31518; A61M 2202/0007; A61M 2202/04; A61M 2205/12; A61M 2205/3327; A61M 2205/50

(57) ABSTRACT

In one example, a drug delivery system, such as an on-body or off-body delivery system is configured to deliver a therapeutic into a patient. The system has a curved track, a plunger, and a driver. The driver causes the plunger to translate along the curved track such that a flexible plunger rod of the plunger bends along the curved track to drive a plunger seal of a drug container to expel a liquid drug from the drug container.

### TECHNICAL FIELD

The present disclosure relates to drug delivery systems, and more specifically, but not necessarily exclusively, to drug delivery systems that deliver a liquid drug.

### BACKGROUND

Pharmaceutical products (including large and small molecule pharmaceuticals, hereinafter “drugs”) are administered to patients using a variety of different drug delivery devices for the treatment of a variety of different medical indications. Drug delivery devices for delivering liquid drugs include, for example, syringes, manual injectors, pen injectors, autoinjectors, on-body delivery devices, and off-body delivery devices. These delivery devices commonly include an actuator, a drug container, and a needle or cannula. The drug container contains the liquid drug and the actuator drives the liquid drug from the drug container, and through the needle or cannula to the patient.

### BRIEF DESCRIPTION OF THE DRAWINGS

The following description of the illustrative embodiments may be better understood when read in conjunction with the appended drawings. It is understood that potential embodiments of the disclosed systems and methods are not limited to those depicted.

FIG. 1 shows a simplified schematic diagram of a drug delivery system according to one example;

FIG. 2 shows a perspective view of a drug container according to one example that can be implemented with the drug delivery system of FIG. 1;

FIG. 3 shows a perspective view of the drug delivery system of FIG. 1 according to one example with a closure in a closed position;

FIG. 4 shows another perspective view of the drug delivery system of FIG. 1 according to one example with the closure in an open position and a container in an uninstalled position;

FIG. 5 shows a side view of the drug delivery system of FIG. 1 according to one example;

FIG. 6 shows a perspective view of an actuator and track according to one example that may be used to implement an actuator and track of the drug delivery system of FIG. 1;

FIG. 7 shows a plan view of the actuator of FIG. 6 with at least a portion of the track removed;

FIG. 8 shows a perspective view of a portion of the actuator of FIG. 6 that includes a flexible plunger rod;

FIG. 9 shows an enlarged perspective view of a portion of the flexible plunger rod of FIG. 8;

FIG. 10 shows a cross-sectional view of a portion of the flexible plunger rod of FIG. 8;

FIG. 11 shows a perspective view of flexible plunger rod and track according to another example that may be used to implement the flexible plunger rod and track of the drug delivery system of FIG. 1;

FIG. 12 shows a perspective view of flexible plunger rod according to yet another example that may be used to the plunger rod of the drug delivery system of FIG. 1;

FIG. 13 graphically illustrates exemplary delivery forces needed to deliver a 100 centipoise (cP) fluid and a 326 centipoise fluid at different flow rates and different needle gauges from a drug container using a straight plunger rod;

FIG. 14 graphically illustrates a performance envelope observed for a prototype drug delivery device of this disclosure;

FIG. 15 shows a sectional view of a portion of the drug delivery system according to another example;

FIG. 16A shows a sectional view of the drug delivery system of FIG. 15 according to one example with a drug container in a pre-pierced position;

FIG. 16B shows a sectional view of a portion of the drug delivery system of FIG. 15 according to one example with a drug container in a pierced position;

FIG. 17A shows a perspective view of a portion of the drug delivery system according to another example with a needle shield in a shielded position;

FIG. 17B shows a perspective view of a portion of the drug delivery system of FIG. 17A with the needle shield in an exposed position;

FIG. 18 shows schematic views of the drug delivery system of FIG. 15 according to one example with a drug container moving between the pre-pierced position, the pierced position, and a removed position;

FIG. 19 shows a schematic view of the drug delivery system of FIG. 15 according to one example illustrating a position of the drug container relative to a septum piercing needle and a plunger;

FIG. 20 shows another schematic view of the drug delivery system of FIG. 15 according to one example illustrating the position of the drug container relative to the septum piercing needle and the plunger; and

FIG. 21 shows an exemplary method of using the drug delivery system according to one example.

### DETAILED DESCRIPTION OF ILLUSTRATIVE EMBODIMENTS

The present disclosure relates to on-body delivery systems (OBDSs) and off-body delivery systems that are configured to inject a liquid therapeutic (e.g., drug or pharmaceutical) into a patient. Although some existing on-body delivery systems are in various stages of commercial development, the inventors have found that these existing systems might not meet the needs of some future therapeutics, particularly the needs of some future large molecule (e.g., biologic) therapeutics. Some of these therapeutics might require systems that are capable of delivering the therapeutic to the patient with significantly higher driving forces that exceed the capabilities of existing on-body delivery systems. For example, some future therapeutics may have relatively high viscosities (discussed further below) that require higher driving forces to deliver the therapeutics. The need for higher driving forces may also be dictated by a need for subcutaneous injections, a need for relatively fast flow rates, and a need for relatively short injection times. These future therapeutics may also require the ability to deliver multiple doses of the same therapeutic, separate doses of different therapeutics, and/or variable volume doses based on, for example, patients' weight and/or age. The present application relates to drug delivery systems, and features thereof, that address various needs of future therapeutics.

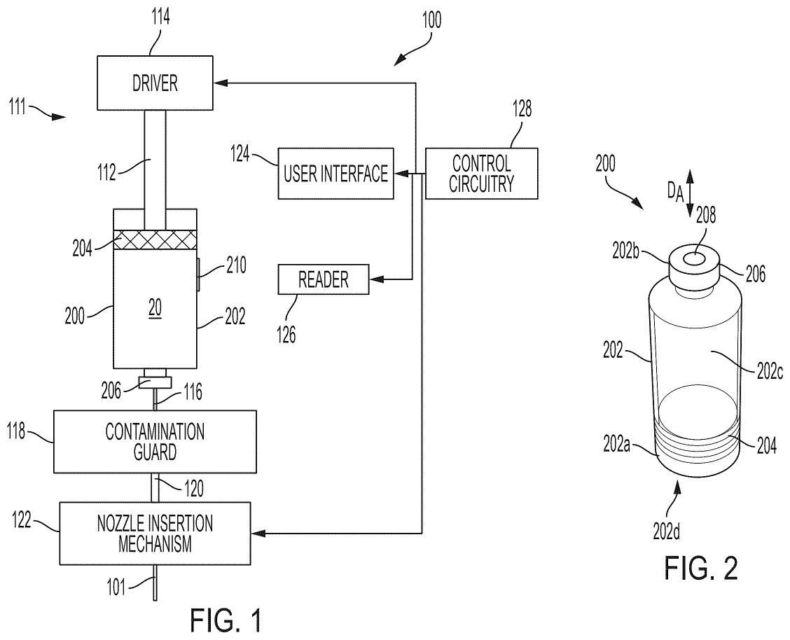

Referring to FIG. 1, a simplified schematic of a drug product according to one example is illustrated. The drug product comprises a drug delivery system 100 and a liquid therapeutic 20 contained within the drug delivery system 100. The drug delivery system 100 can be a prefilled drug delivery system 100 distributed with the therapeutic 20 contained therein such that the user (e.g., healthcare provider or patient) need not fill the drug delivery system 100 with the therapeutic 20 prior to use. In alternative examples, the drug delivery system 100 can be distributed separately from the therapeutic 20 such that the drug delivery system 100 needs to be filled with the therapeutic 20 prior to use.

The drug delivery system 100 is configured to expel the liquid therapeutic 20 from a drug container 200 and into a patient via a nozzle 101. The nozzle 101 is configured to be inserted into a patient such as into a patient's skin. The nozzle 101 can be, for example, a needle or cannula. Preferably, the drug delivery system 100 is a subcutaneous delivery system configured to deliver the therapeutic 20 to a subcutaneous layer of the patient's skin. Thus, the nozzle 101 can configured to extend from the system 100 by a distance that extends into, but not beyond, the subcutaneous layer. This distance can be in a range of, for example, about 6 mm to about 8 mm.

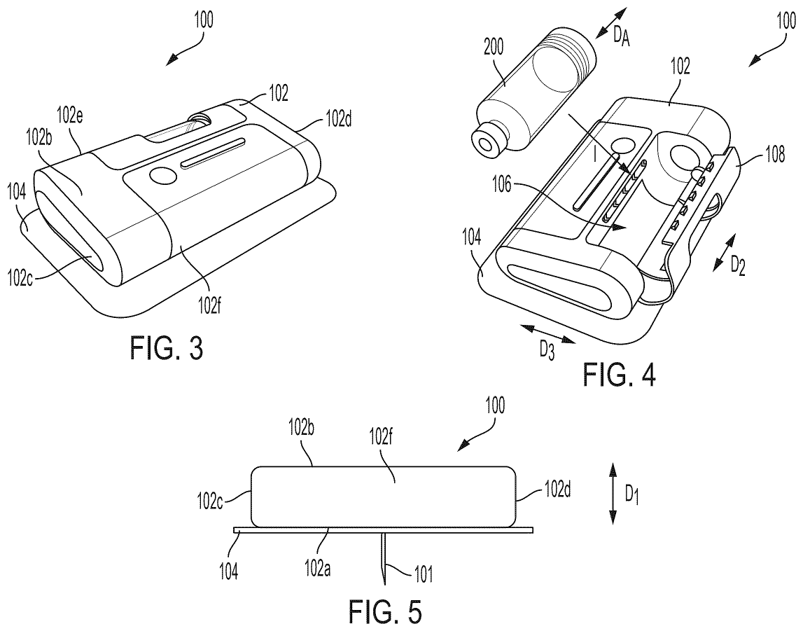

The drug delivery system 100 can be used as an on-body delivery system (OBDS), where the drug delivery system 100 abuts the patient's body. In such examples, the nozzle 101 can extend from a housing (e.g., 102 of FIGS. 3 to 5 below) of the drug delivery system 100 into the patient, where the housing abuts the patient. Alternatively, the drug delivery system 100 can be used as an off-body delivery system, where the housing of the drug delivery system 100 is spaced from the nozzle when the nozzle is inserted into the patient. In such examples, the drug delivery system 100 can include conduits (e.g., tubing) that span a gap between the housing of the drug delivery system 100 and the nozzle 101 to route the drug from the drug delivery system 100 to the nozzle 101. Alternatively still, the drug delivery system 100 can be selectively configurable to be used as either an on-body delivery system or an off-body delivery system.

The drug delivery system 100 can comprise the drug container 200 or the drug container 200 can be a separate component from the drug delivery system 100. The drug container 200 can be supported by, or configured to be supported by, a housing of the drug delivery system 100. In some examples, the drug container 200 can be removably attachable to or removably insertable into the housing of the drug delivery system 100. In other examples, the drug container 200 can be fixedly attached to or fixedly inserted into the housing. In yet other examples, the drug container 200 can be integral with the housing.

The drug container 200 can be any suitable container for containing a liquid drug, such as a cartridge or a syringe. FIG. 2 shows an example of a drug container 200 according to one example, where the drug container 200 is a cartridge. The drug container 200 comprises a container body 202 defining a cavity 202c configured to hold a liquid drug therein. The container body 202 has a first end 202a and a second end 202b. The container body 202 can have a central axis that extends along an axial direction D_{A}. The first end 202a can define an opening 202d therein that is open to the cavity 202c. The drug container 200 can comprise a seal 204 disposed in the cavity that forms a seal with an interior surface of the container body 202. The seal 204 can be received through the opening 202d into the cavity 202c. The seal 204 is configured to translate towards the second end 202b to drive the liquid drug from the cavity 202c.

In some examples, such as where the drug container 200 is a cartridge, the drug container 200 can comprise a cap 206 on the second end 202b. The cap 206 can be formed from any suitable material, such as a metal. The cap 206 can be crimped onto a head of the container body 202 at the second end 202b. The drug container can comprise a septum 208 supported by the cap 206. The septum 208 is configured to seal the second end 202b. The septum 208 is configured to be pierced by a piercing needle to open a fluid path into the drug container 200. The septum 208 can optionally be configured to reseal the second end 202b when the piercing needle is removed from the septum 208. The space between the cap 206 and the seal 204 is filled with a substantially incompressible fluid (e.g., the therapeutic and possibly air) that prevents movement of the seal 204 until the septum 208 is pierced.

Returning to FIG. 1, the drug delivery system 100 comprises an actuator 111 configured to drive a liquid therapeutic 20 from the drug container 200 out of the needle or cannula 101. The actuator 111 can be any suitable actuator for expelling a liquid therapeutic 20 from the drug container 200. The actuator 111 can comprise a plunger 112 that is configured to move the seal 204 of the drug container 200 to drive a liquid therapeutic 20 from the drug container 200. The actuator 111 can comprise a driver 114 that is configured to cause the plunger 112 to move the seal 204. The driver 114 can be any suitable driver, such as (without limitation) a motor, a spring, a hydraulic driver, or a pneumatic driver. The plunger 112 can be any suitable plunger, such as a flexible plunger or a telescoping plunger.

The drug delivery system 100 can comprise a septum piercing needle 116 that is configured to pierce the septum 208 of the drug container 200. At least one of the septum piercing needle 116 and the drug container 200 can be configured to move towards the other to cause the piercing needle 116 to pierce the septum 208. Piercing the septum 208 can place the septum piercing needle 116 in fluid communication with the liquid therapeutic 20 contained within the drug container 200. The drug delivery system 100 can comprise a conduit 120, such as tubing, that fluidly connects the septum piercing needle 116 to the nozzle 101. Thus, piercing the septum 208 can place the nozzle 101 in fluid communication with the liquid therapeutic 20 contained within the drug container 200 via the septum piercing needle 116 and the conduit 120.

The drug delivery system 100 can optionally comprise a contamination guard 118 that protects the piercing needle 116 from contamination when the piercing needle 116 is not piercing the septum 208 of the drug container 200. For example, the contamination guard 118 can be configured to protect the piercing needle 116 from contamination before the drug container 200 is supported by a housing of the drug delivery system 100, while the drug container 200 is supported by the housing but before the septum 208 is pierced, and/or after the drug container 200 is removed from the housing post injection.

The drug delivery system 100 can comprise a nozzle insertion mechanism 122 that is configured to cause the nozzle 101 to be inserted into a patient, such as into a patient's skin. The nozzle insertion mechanism 122 can be configured to cause the nozzle 101 to extend out of a housing of the drug delivery system 100 and into the patient. In some examples, the nozzle insertion mechanism 122 can be configured to cause the nozzle 101 to retract back into the housing after injection. Additionally, or alternatively, the drug delivery system 100 can comprise a needle guard (not shown) that extends over the nozzle 101 after injection. Retracting and/or covering the nozzle 101 after injection can prevent inadvertent needle sticks and/or limit human contact with biological materials remaining on the nozzle 101. The nozzle insertion mechanism 122 can be any suitable mechanism, including (without limitation) those known in the art, for inserting the nozzle 101 into a patient. The nozzle insertion mechanism 122 can include a driver, such as a motor or spring, that causes the nozzle 101 to be inserted into the patient.

The drug delivery system 100 can comprise control circuitry 128 that is configured to control various features of the drug delivery system 100. For example, the control circuitry 128 can be configured to control operation of the driver 114 of the actuator 111. The control circuitry 128 can be configured to cause the actuator 111 to begin driving the liquid therapeutic 20 from the drug container 200. The control circuitry 128 can be configured to control the flow rate in which the liquid therapeutic 20 is driven from the drug container 200. The control circuitry 128 can be configured to cause the actuator 111 to stop driving the liquid therapeutic 20 when an injection is complete and/or when an error is detected during the injection. The control circuitry 128 can also be configured to control operation of the nozzle insertion mechanism 122 to cause the nozzle 101 to be inserted into the patient before injection and/or removed from the patient after injection.

The drug delivery system 100 can comprise a user interface 124 that is configured to be engaged by a user such as a health care provider or patient to operate the drug delivery system 100. The user interface 124 can be configured to provide information to a user of the drug delivery system 100. The user can be, for example, a patient, or a patient's care giver or health care professional assisting the patient in using the drug delivery system 100. The user interface 124 can have a variety of configurations, and the drug delivery system 100 can include a single type of user interface or can include more than one type of user interface. For example, the user interface 124 can include one or more lights, e.g., a light emitting diode (LED) or other type of light, configured to illuminate to provide various information. Examples of the information indicated by the user interface 124 include power (on/off) status, error state (e.g., low power supply, improper nozzle advancement into the patient, incompatible type of container 200 loaded into the drug delivery system 100, etc.), drug delivery status (e.g., indication that drug delivery is currently occurring), drug delivery progress information, an orientation of the drug delivery system 100 relative to gravity, an indication of a dose of the drug 20 to be provided in each delivery of the drug 20 to the patient, and other types of information.

For another example, the user interface 124 can include a display configured to show information thereon, such as by using text and/or graphics. The display can include a display screen having any of a variety of configurations, such as a cathode ray tube (CRT), a liquid crystal display (LCD), a touchscreen, etc. For yet another example, the user interface 124 can include a vibration mechanism configured to vibrate with the vibration being configured to be felt by the patient wearing the drug delivery system 100. For still another example, the user interface 124 can include a speaker configured to provide an audio signal. For another example, the user interface 124 can include a mechanical level configured to indicate the pump's orientation.

In some examples, the system can comprise a data storage component 210 supported by the drug container 200 and a reader 126 that is configured to read the data storage component 210. The reader 126 can be supported by, for example, the housing 102. The data storage component 210 can be attached to drug container 200, such as the body 202 or cap 206 of the drug container 200, such as by being adhered to the drug container 200, or that is otherwise part of the container 200, such as by being printed thereon. The data storage component 210 can have a variety of configurations. For example, the data storage component 210 can include an integrated circuit configured to communicate the reservoir data from the reservoir. One example of an integrated circuit is a near field communication (NFC) tag, also referred to as a proximity-integrated circuit card (PICC). An ISO14443 A passive NFC tag, an ISO15693 passive NFC tag, an ISO18000-3 passive NFC tag, an ISO14443 A/B passive NFC tag, a passive FeliCa NFC tag, or other type of NFC tag (passive or active) can be used. For another example, the data storage component 210 can include a radio frequency identification (RFID) tag. For yet another example, the data storage component can be in the form of a barcode. One example of a barcode is a QR code. Another example of a barcode is a Universal Product Code (UPC) code.

The drug container 200 includes a single data storage component 210 in this illustrated embodiment but can include a plurality of data storage components. If a plurality of data storage components are used, each can be different from one another, which may help provide redundancy and/or allow for data retrieval even if a certain type of data communication is currently unavailable, e.g., if an RFID tag is absent or damaged so as to be unreadable a QR code may still be read.

The data storage component 210 is configured to store data regarding the drug container 200, regarding the drug 20 contained in the drug container 200, and/or regarding delivery parameters of the drug 20. For example, the data storage component 210 can store information related to a dosing regimen of the drug 20 in the drug container 200. For instance, the data storage component can store one or more of: the volume of the drug 20 stored in the drug container 200, the amount of drug 20 to be delivered (dose amount) by the system 100 (which can be less than the amount stored in the container 200), the flow rate in which the system 100 is to deliver the drug 20, or any suitable data for configuring an operating parameter of the system 100.

In some examples, the reader 126 can transmit data as well as receive data, and optionally cause that data to be written to the data storage component 210. In some such examples, the data storage component 210 can be updated at regular intervals (e.g., at delivery completion of each mL) so that it contains a reasonably-accurate record of the delivery progress at any given time. If the system 100 should fail during delivery, resulting in a partially-delivered dose, the reservoir containing a record of the partial dose can be transferred to a secondary system, where the remaining dose could be delivered. In some such examples, the data storage component 210 can also be updated with any information relevant to the delivery and state of the system 100 during delivery.

For example, the information can include the date and time of delivery, the model and serial number of the system 100, the ambient system and container temperatures, system user input settings, system wireless communications events, system warning or alarm events, user-initiated pauses and durations, user interface events, and/or relevant system parameters settings and measurements during delivery (force, pressure, battery voltage/current, etc.). As such, the data storage component 210 could serve as a record of the delivery (e.g., a delivery “black-box” recording). The data storage component 210 can be designed to easily peel off the reservoir, so that it can be transferred to a monitoring party or HCP for subsequent reading, recording and analysis. Upon completion of delivery, the data storage component 210 can be updated with a “delivery-completed” status, thus preventing the reservoir from being refilled and reused.

The control circuitry 128 is configured to control administration of the drug 20 from the system 100 according to a dosing regimen. This can be accomplished using the data read by the reader 126 from the data storage component 210 and/or data stored in the control circuitry 128. The dosing regimen refers to the specific manner in which the drug is delivered, including (without limitation) formulation, route of administration, dose interval (frequency of dosing), dose amount or volume, delivery rate (flow rate), delivery duration, pauses in delivery, pauses between delivery phases in a multi-drug delivery sequence, and sequencing order of a multi-drug delivery sequence. The dosing regimen can be stored as an algorithm in a memory of the control circuitry 128 that a processor of the control circuitry 128 is configured to execute. The algorithm is stored in the form of one or more sets of pluralities of data points defining and/or representing instructions, notifications, signals, etc. to control administration of a drug from the system 100.

Turning to FIGS. 3 to 5, a housing 102 is shown according to one example that can be used to implement a housing of the drug delivery system 100 of FIG. 1. In examples where the drug delivery system 100 is an on-body system, the drug delivery device housing 102 can support, such as house, the actuator 111, the piercing needle 116, the contamination guard 118, the conduit 120, the nozzle insertion mechanism 122, the control circuitry 128, and the user interface 124. In alternative examples, where the drug delivery device is an off-body system, the housing 102 can support, such as house, the actuator 111, the piercing needle 116, the contamination guard 118, the control circuitry 128, and the user interface 124, while the conduit 120 can extend from and outside the housing 102 to the nozzle insertion mechanism 122 that is physically separate from the housing 102.

The housing 102 can be configured to support, such as house, at least a portion up to an entirety of the drug container 200 therein. The housing 102 can be configured to receive, and in some cases removably receive, the drug container 200. Thus, the drug container 200 can be insertable into and/or removable from the housing 102. The housing 102 can define an opening 106 therein that is configured to receive the drug container 200 at least partially or fully into the housing 102. In some examples, the housing 102 can comprise a closure 108, such as a door, that is configured to close at least a portion of the opening 106 to maintain the drug container 200 in the opening 106. The opening 106 can be configured such that the drug container 200 is received into the opening 106 along an insertion direction I. The insertion direction I can be transverse to a longitudinal axis of the drug container 200 and/or a longitudinal axis of the opening 106.

The drug delivery device housing 102 can have a bottom 102a and an opposing top 102b that are opposite from one another along a first direction D₁. In this example, the drug delivery system 100 is an on-body delivery system, and the drug delivery system 100 is configured such that the bottom 102a faces the skin of the patient when the drug delivery system 100 is attached to the patient. The housing 102 can have a first side 102c and a second side 102d opposite from one another along a second direction D₂. The first and second sides 102c and 102d can extend between the bottom 102a and the top 102b. The housing 102 can have a first end 102e and a second end 102f that are opposite one another along a third direction D₃. When the drug container 200 is received in the opening 106, the central axis of the drug container 200 can extend along the second direction D₂. Thus, the axial direction D_{A} can be substantially aligned with the second direction D₂. The first and second ends 102e and 102f can extend between the first and second sides 102c and 102d and between the bottom 102a and bottom 102b. The opening 106 extends into the top 102b and the first end 102e. In alternative examples, the opening can extend into another suitable surface, such as one or more of the bottom 102a, the top 102b, the first end 102e, and the second end 102f.

In this example, the drug delivery system 100 can be used as an on-body delivery system. Thus, the drug delivery system 100 comprises a fastener 104 configured to attach the housing 102 to a patient's body. The fastener 104 can be any suitable fastener for attaching to a patient's body, such as (without limitation) an adhesive, including a tape with adhesive, a strap, or other suitable fastener. The fastener 104 can be supported by the bottom 102a of the housing 102. Additionally, or alternatively, drug delivery system 100 can be used as an off-body delivery system. In such alternatives, the drug delivery system 100 might not employ the fastener 104.

### Drug Delivery System Actuator

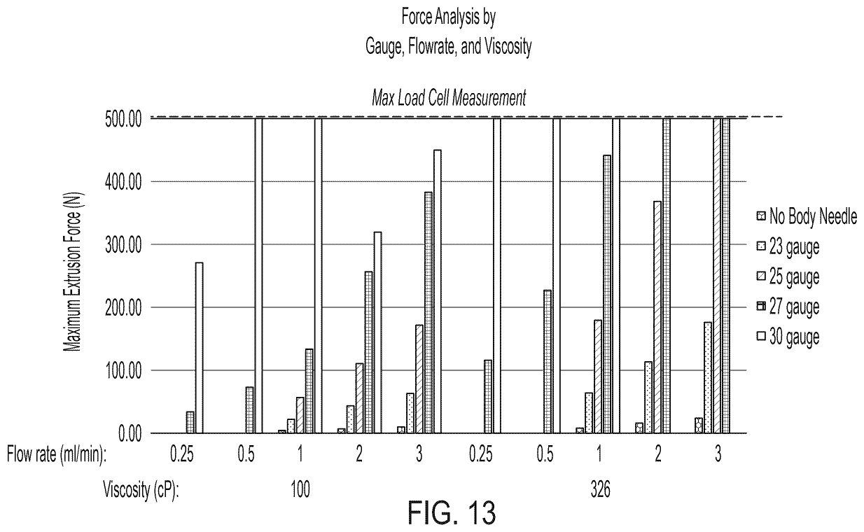

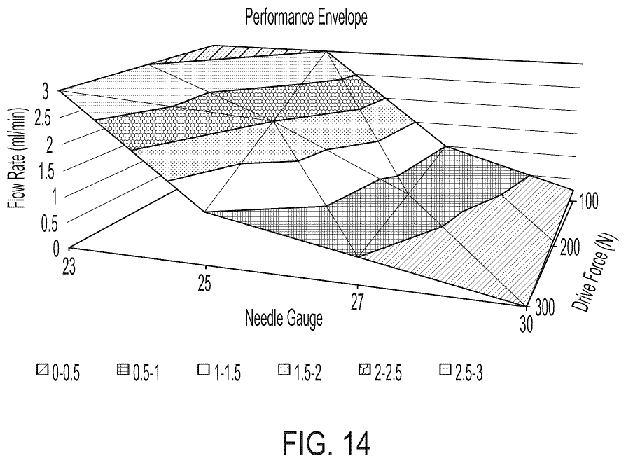

The ability of an on-body delivery system or off-body delivery system to deliver a liquid drug into a patient is dependent on several parameters, including viscosity of the drug, drug type (e.g., solution or suspension), particle size of the drug, needle gauge, and flow rate. Variation of one or more of these parameters can significantly increase or decrease the amount of drive force needed to deliver the liquid drug. Thus, for a given needle gauge and flow rate, higher driving forces are typically needed to deliver higher viscosity drugs (e.g., 100 cp, 200 cp, 300 cp, or even 400 cp), while lower driving forces are typically needed to delivery lower viscosity drugs (e.g., <100 cp). For a given viscosity and flow rate, higher driving forces are typically needed to deliver the drug through a needle having a larger needle gauge, while lower driving forces are typically needed to deliver the drug through a needle having a smaller needle gauge. For a given viscosity and needle gauge, higher driving forces are typically needed to deliver the drug at a higher flow rate, while lower driving forces are typically needed to deliver the drug at a lower flow rate. This problem can be exacerbated when two or more of these factors are varied. For instance, even higher driving forces are typically needed to deliver a higher viscosity drug through a larger gauge needle at a higher flow rate. The forces needed to deliver drugs by varying these parameters can exceed 50N, 75N, 100N, 150N, 200N, 250N, 300N, 350N, or even 400N. This is illustrated by FIG. 13, which shows exemplary delivery forces needed to deliver a 100 centipoise (cP) fluid and a 326 centipoise fluid at different flow rates and different needle gauges from a drug container using a straight plunger rod.

Conventional on-body delivery systems are not typically designed to deliver drugs with such high forces. Rather, conventional on-body delivery systems commonly have driving mechanisms that deliver liquid drugs using relatively low driving forces (e.g., ≤30 N). This may be due in part to a lack of need for on-body delivery systems with higher driving forces and/or size and weight constraints of on-body delivery systems. For instance, on-body delivery systems tend to be used with lower viscosity drugs that do not require higher driving forces. Difficulties in delivering these lower viscosity drugs can be often resolved by simply decreasing the flow rate of an existing on-body delivery systems or decreasing the gauge (i.e., increasing the diameter) of the needle of an existing on-body delivery systems. However, decreasing needle gauge can increase patient discomfort.

Conventional on-body delivery systems might also lack higher driving forces due to size and weight preferences of on-body delivery systems. On-body delivery systems are adhered to, or otherwise supported by, the patient's body. Therefore, it is desirable for on-body delivery systems to be light weight and compact in size for patient comfort. However, increasing the driving forces of an on-body delivery system may require increasing the size, and consequently the weight, of the driver used to drive the on-body delivery system to such an extent that the on-body delivery system is no longer suitable for on-body use. In on-body delivery systems in which the drive mechanism is electromechanical, increasing the size of the drive mechanism (e.g., motor) can also require increasing the size of other components, such as a power source (e.g., battery) that powers the on-body delivery system, to accommodate the increased driving forces.

More recently, there has been some interest in delivering drugs having higher viscosities (e.g., 100 cp, 150 cp, 200 cp, 250 cp, 300 cp, 350 cp or even 400 cp) and delivering drugs with larger gauge needles (e.g., 23 gauge, 24 gauge, 25 gauge, 26 gauge, 27 gauge, 28 gauge, 29 gauge, or even 30 gauge) for patient comfort. To satisfy these interests, there is a need for on-body delivery systems that are capable of delivering drugs with higher driving forces (e.g., 50N, 75N, 100N, 150N, 200N, 250N, 300N, 350N, or even 400N), and yet are light weight and compact in size for patient comfort.

In preferred examples, the actuator 111 of FIG. 1 is capable of driving liquid drugs from the drug container 200 with higher driving forces above 30N, such as above one of 50N, 75N, 100N, 125N, 150N, 175N, 200N, 225N, 250N, 275N, 300N, 325N, 350N, 375N, or 400N. Note that the actuator 111 may still be capable of driving lower forces less than the aforementioned values. The actuator 111 is capable of delivering drugs having higher viscosities such as 100 cp, 125 cp, 150 cp, 175 cp, 200 cp, 225 cp, 250 cp, 275 cp, 300 cp, 325 cp, 350 cp, 375 cp, or 400 cp, in addition to, or alternatively to, lower viscosities less than any of the aforementioned values. Further, the actuator 111 is capable of delivering drugs with larger gauge needles such as 23 gauge, 24 gauge, 25 gauge, 26 gauge, 27 gauge, 28 gauge, 29 gauge, or 30 gauge needles, at lower or higher viscosities. FIG. 14 graphically illustrates a performance envelope observed for a prototype drug delivery device of this disclosure. In this example, a 400N force is applied to a plunger 302 (discussed below), and the surface represents the upper limit of the delivery capabilities of the device at various flow rates, needle gauges, and viscosities.

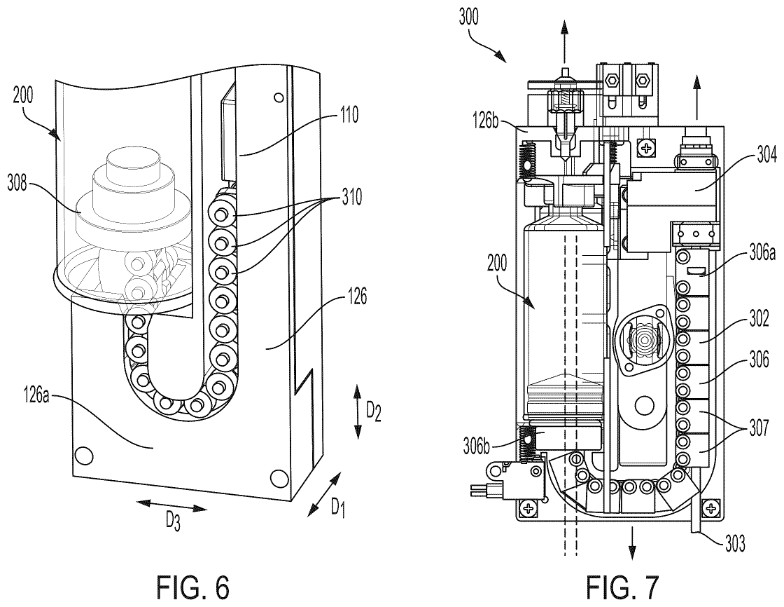

Referring to FIGS. 6 and 7, internal features of the drug delivery system 100 are shown, including one example of an actuator 300 that can be used to implement the actuator 111 of the drug delivery system 100. The drug delivery system 100 comprises at least one track 110, and the actuator 300. The actuator 300 comprises a plunger 302 that is configured to be guided by the at least one track 110. The plunger 302 can implement the plunger 112 of FIG. 1. The plunger 302 can be rotationally and/or torsionally fixed relative to a central axis of the plunger 302. Note that the central axis can be curved along a length of the plunger 302.

The actuator 300 comprises a driver 304 that is configured to cause the plunger 302 to translate within the drug container 200 to drive the seal 204 of the drug container 200 to expel the liquid drug from the drug container 200. The driver 304 can implement the driver 114 of FIG. 1. The driver 304 can be any suitable driver that can drive the plunger 302, such as (without limitation) a motor, a spring, a pneumatic actuator, a hydraulic actuator, or an electric actuator. In a preferred example, the driver 304 comprises a motor and the actuator 300 comprises a threaded rod 303. The threaded rod 303 can extend inside at least a portion of the plunger 302 and engage internal threads of the plunger 302. The actuator 304 can be configured such that, when the motor rotates the threaded rod 303, the threads of the threaded rod 303 engage the threads of the plunger 302, thereby causing the plunger 302 to translate within the drug container 200.

The plunger 302 can have a flexible plunger rod 306, a first plunger end 306a and a second plunger end 306b. The second plunger end 306b is configured to engage the seal 204 of the drug container 200. The flexible plunger rod 306 is configured to bend as it drives the second plunger end 306b. The flexible plunger rod 306 can comprise a plurality of links 307 (as shown) that are pivotably connected to one another. In other examples, the flexible plunger rod 306 can additionally or alternatively comprise a flexible material that is capable of bending (e.g., an elongate bar made from a flexible material that bends). The first plunger end 306a can be configured to engage the threaded rod 303. For example, the first plunger end 306a can define the internal threads that engage the threaded rod 303. It will be understood that, in alternative examples, the plunger 302 can be driven by a mechanism other than the motor 304 and threaded rod 303, such as by a magnetic drive.

The at least one track 110 can define a curved path that guides the plunger 302 to bend within a range from 45 degrees to 225 degrees, such as about 90 degrees or preferably about 180 degrees. In some examples, the track 110 can define a U-shaped or J-shaped path for the plunger rod 306. The track can be defined by a recess or opening as shown. Alternatively, the track can be defined by a rail. The flexible plunger rod 306 is configured to bend within the range from 45 degrees to 225 degrees as it is guided around the track 110. By employing the flexible plunger rod 306 and curved track 110, the distance that the plunger rod 306 extends out behind the drug container 200 is significantly reduced compared to comparable devices in which the plunger rod extends straight out behind the drug container. As a result, the overall length of the drug delivery system 100 can be less than that of such comparable devices, resulting in system that is more compact for patient comfort.

Friction resulting from the flexible plunger rod 306 bending along the curved track 110 can result in significant losses of force between the driver 304 and the point where the plunger 302 engages the seal 204 of the drug container 200. The loss can be, for example, a loss of 50 percent of the force or greater. In order to drive a liquid drug from the drug container 200 with the higher driving forces mentioned above, the size of the driver 304 can be increased. However, increasing the size of the driver 304 (and size of associated parts such as power sources) increases patient discomfort. In fact, the size of the driver 304 may need to be increased to such an extent that it would be incompatible for use in an on-body delivery system.

Instead of increasing the size of the driver 304, the interface between the plunger rod 306 and track 110 can be implemented with friction reduction so that a force needed to translate the plunger seal 204 within the container 200 via the plunger rod 306 is no greater than 30%, such as no greater than 25%, 20%, 15%, 10%, or 5% more than a force that would be needed to translate the plunger seal within the container with a straight plunger rod. Use of the friction reduction at the interface between the plunger rod 306 and the track 110 can enable the drug delivery system 100 to be implemented with smaller drivers (and hence smaller power sources) that are more compatible with on-body use, while still having the ability to drive the larger forces discussed above. In some examples, the interface can be implemented with a friction reduction coating that reduces friction between the plunger rod 306 and the track 110. In other examples as shown in FIGS. 6 to 12, the interface can comprise friction reduction members 310 such as rollers or bearings that reduce friction at the interface.

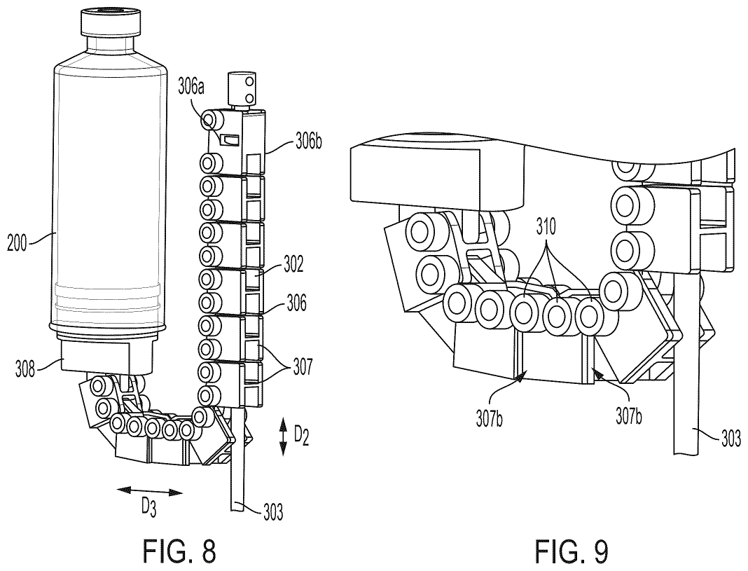

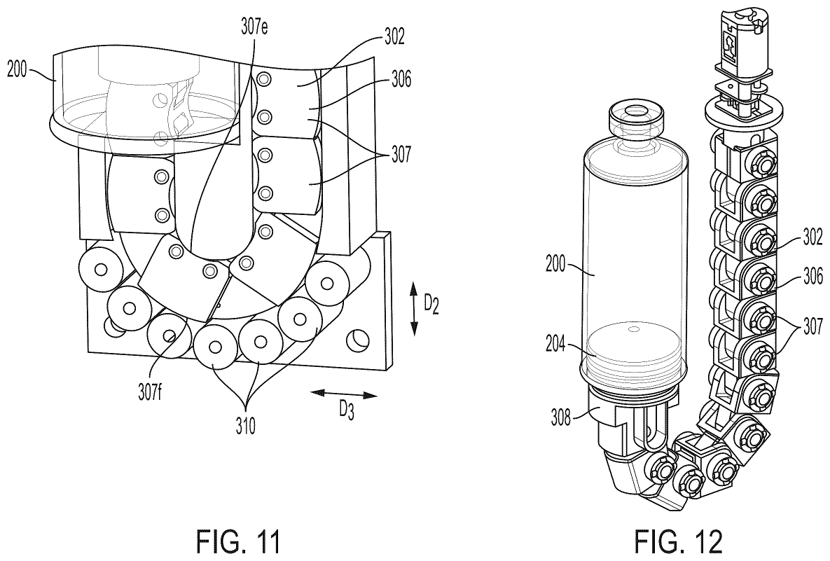

For instance, the drug delivery system 100 can comprise at least one roller or bearing 310 configured to guide the flexible plunger rod 306 as the flexible plunger rod 306 translates along the curved track 110 to limit any loss in force. The flexible plunger rod 306 can support the least one roller or bearing 310 (see e.g., FIGS. 8 to 10) such that the least one roller or bearing 310 moves with the flexible plunger rod 306 relative to (e.g., along) the track 110. Alternatively, the track 110 can support the least one roller or bearing 310 (see e.g., FIG. 11) such that plunger rod 306 moves relative to (e.g., along) the at least one roller or bearing 310 and the track 110.

With continued reference to FIGS. 6 to 9, the flexible plunger rod 306 can have a first outboard side 306a and a second outboard side 306b. The first and second outboard sides 306a and 306b can be opposite from one another along the first direction D₁. The at least one roller or bearing 310 can comprise one or more rollers or bearings 310 disposed on the first outboard side 306a of the flexible plunger rod 306. In some examples, the at least one roller or bearing 310 can comprise one or more rollers or bearings 310 disposed on the second outboard side 306b of the flexible plunger rod 306. In such examples, the at least one track 110 can comprise a pair of tracks 110. The pair of tracks 110 can be opposite from one another along the first direction D₁. The one or more rollers or bearings 310 of the first outboard side 306a can ride along a first one of the tracks 110, and the one or more rollers or bearings 310 of the second outboard side 306b can ride along a second one of the tracks 110. In alternative examples, the at least one roller or bearing 310 can be disposed between the first and second outboard sides 306a and 306b.

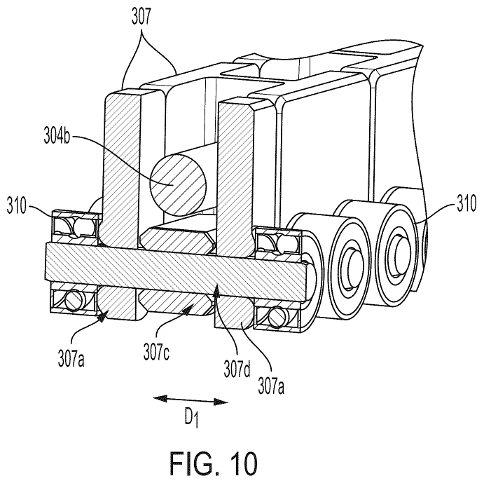

In examples with links 307, such as shown in FIGS. 8 to 10, each link 307 can comprise opposing sides 307a, and opposing ends 307b. The opposing sides 307a can be opposite one another along the first direction D₁. The opposing sides 307a can extend between the opposing ends 307b. The links 307 can be disposed adjacent one another such that the opposing ends 307b are arranged end-to-end along the length of the plunger rod 306. Each adjacent pair of links 307 can be connected by a connector 307c. In some examples, each connector 307c can be pivotably coupled to an adjacent pair of links 307 as shown in FIG. 10. In other examples, each connector 307c can be fixedly attached to one end 307b of a respective link 307 and can be received between the opposing sides 307a of an adjacent link 307 as shown in FIG. 12. It will be understood that other configurations of links are contemplated within the scope of this disclosure.

The at least one roller or bearing 310 can be supported by, such as attached to, each of one or more of the links 307, up to all of the links 307. Each roller or bearing 310 can be supported outboard of a side 307a of a respective link 307 as shown. In other examples (not shown), each roller or bearing 310 can be supported between opposing sides 307a of a link 307. In some examples, each respective link 307 can support at least one pair of rollers or bearings 310. The rollers or bearings 310 of each pair can be supported on opposing sides 307a of a respective link 307. Each roller or bearing 310 is configured to roll along the track 110 to limit friction between the plunger rod 306 and the track 110. Each roller or bearing 310 can be supported by a respective axle 307d that extends from or through a respective link 307.

Turning to FIG. 11, in an alternative example, the at least one roller or bearing 310 can be positionally fixed relative to the housing 102 of the drug delivery system 100, and the plunger 306 can be configured to move relative to and along the at least one roller or bearing 310. Each link 307 of the plunger 306 can have an inner end 307e and an outer end 307f that are opposite one another. The inner end 307e and outer end 307f of each link can be opposite one another in a plane defined by the second direction D₂ and third direction D₃. The inner ends 307e can face inwards to define a curve at a bend in the plunger 306 that has a first radius. The outer ends 307f can face outwards to define a curve having at the bend that has a second radius, greater than the first radius. The at least one roller or bearing 310 can be configured to engage the outer ends 307f of the links 307 of the plunger 306. The at least one roller or bearing 310 can disposed along the curve defined by the track 110.

Referring back to FIGS. 6 and 7, driving a liquid drug from the drug container 200 with the higher driving forces mentioned above can exert significant opposing forces on the drug delivery system 100, and particularly, on the housing 102. These opposing forces can be applied by the plunger rod 306 at the curve defined by the track 110 at one end, and by the drug container 200 and/or driver 304 at the other end as indicated by the arrows in FIG. 7. These forces can be so significant that, without reinforcements, the forces can cause the housing 102 to burst. Therefore, the drug delivery system 100 can comprise reinforcement structure 126 that is configured to absorb at least some, up to all, of the opposing forces. The reinforcement structure 126 is configured to limit or prevent the opposing forces from being exerted on the housing 102. In some examples, the reinforcement structure can comprise a rigid plate formed from a suitably rigid material such as metal. The reinforcement structure 126 can have a first end 126a that resists outward movement of the at least one curved track at a first end of the drug delivery system along a select direction (e.g., downwards in FIGS. 6 and 7), and a second end 126b that resists outward movement of the drug container and/or driver at a second end of the drug delivery system along a direction (e.g., upwards in FIGS. 6 and 7) opposite the select direction.

The reinforcement structure 126 can define the at least one track 110. For example, the at least one track 110 can be defined by an opening or recess that extends into or through the reinforcement structure 126. The opening or recess can be configured to receive the at least one roller or bearing 310 therein. In some examples, the reinforcement structure 126 can be disposed on opposing sides of the plunger rod 306. For example, the reinforcement structure 126 can comprise a pair of opposing rigid plates disposed on opposing sides of the plunger rod 306, where each rigid plate defines a corresponding track 110, each corresponding track 110 configured to receive at least one roller or bearing 310.

With reference to FIGS. 5 to 7, in operation, a method of delivering a drug to a patient with a drug delivery system 100 can comprise inserting a needle or cannula 101 of the drug delivery system 100 into the patient. The method comprises causing a flexible plunger rod 306 of the drug delivery system 100 to translate along at least one curved track 110 of the drug delivery system 100 such that the flexible plunger rod 306 bends as it translates along the at least one curved track 110 into the drug container 200 of the drug delivery system 100 to drive a liquid drug from the drug container 200 into the patient. At least one roller or bearing 310 of the drug delivery system 100 can guide the flexible plunger rod 306 as the flexible plunger rod 306 translates along the at least one curved track 110. The at least one roller or bearing 310 can ride along the at least one curved track 110 with the flexible plunger rod 306 (e.g., FIGS. 6, 7), or the plunger rod 306 can ride along the at least one roller or bearing 310 (e.g., FIG. 11). Additionally or alternatively, the method can comprise causing a flexible plunger rod 306 of the drug delivery system 100 to translate along at least one curved track 110 of the drug delivery system 100 such that the flexible plunger rod 306 bends as it translates along the at least one curved track 110 into the drug container 200 of the drug delivery system 100 to drive a liquid drug from the drug container 200 with a force of at least 50N, such as at least 100 N, 150N, 200N, 250N, 300N, 350N, or 400N. The method can comprise a step of causing a driver 304 to cause the threaded rod 303 to rotate to cause the plunger 306 to translate along the at least one track 110.

### Septum Piercing Assembly

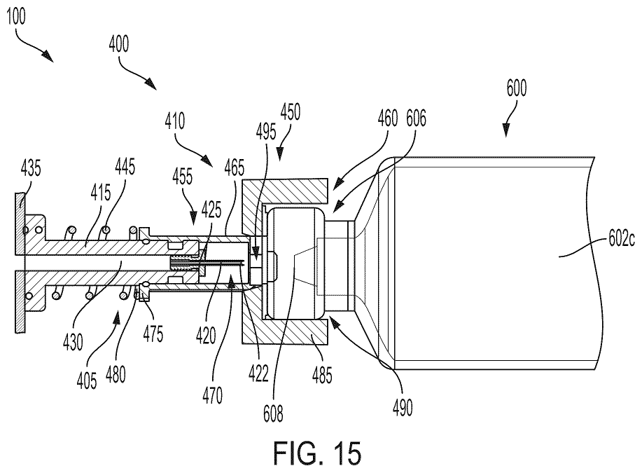

Referring to FIG. 15, internal features of the drug delivery system 100 are shown, including one example of a septum piercing assembly 400. The septum piercing assembly 400 can be configured to removably receive portions of a drug container 600 that can implement and be generally similar to the drug container 200. The septum piercing assembly 400 can implement the septum piercing needle 116 of the drug delivery system 100 The septum piercing assembly 400 can comprise a needle assembly 405 and a biasing assembly 410. The biasing assembly 410 can be movably attached to the needle assembly 405.

The needle assembly 405 can comprise a needle support 415 and a septum piercing needle 420. The septum piercing needle 420 can be received and/or attached to the needle support 415 via any suitable technique such as, for example, a mechanical fastener, a press-fit connection, adhesive, a connecting member, and/or any other suitable technique. For example, the septum piercing needle 420 can be attached to the needle support 415 via a connecting member 425 that can be received in a cavity 430 formed in the needle support 415. The septum piercing needle 420 can include a hollow cavity that can be fluidly connected to the cavity 430 of the needle support 415, which can form a portion of the fluid connection between the septum piercing needle 420 to the nozzle 101 as described above. The septum piercing needle 420 can include a tip 422 for piercing a septum of the drug container 600 as described for example below.

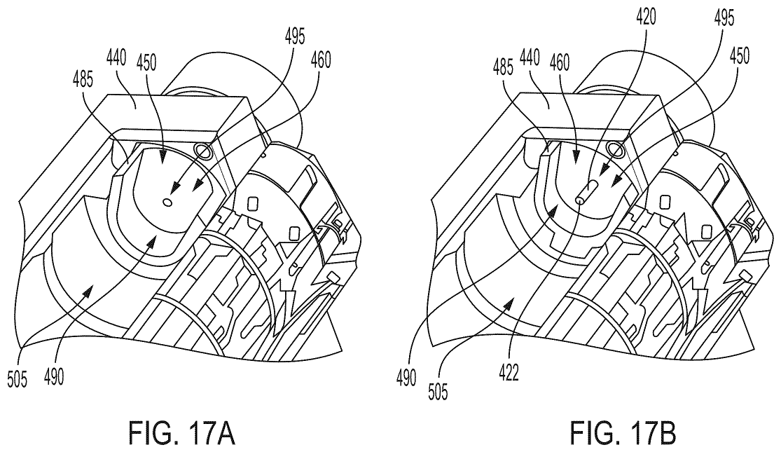

The needle support 415 can be attached to a housing portion 435 of a housing 440, which can implement the housing 102 as further described below regarding FIGS. 17A and 17B. Referring again to FIG. 15, the housing portion 435 can be attached to and/or can be an integral part of the housing 440. The housing portion 435 can be fixedly attached to and/or integrated into the housing 440 so that the needle assembly 405 (e.g., including the septum piercing needle 420) remains substantially stationary relative to the housing 440. The needle support 415 and the connecting member 425 can be formed from material that is compatible with the drug 20. The needle support 415 and the connecting member 425 can be formed from any suitable material such as, for example, structural plastic material (e.g., and the material of the housing 440 may be similar to this material). The septum piercing needle 420 can be formed from any suitable material such as, for example, metal material (e.g., stainless steel).

The biasing assembly 410 can comprise a biasing member 445 and a needle shield 450. The biasing member 445 and the needle shield 450 can be movably supported by (e.g., attached to) the needle support 415 of the needle assembly 405.

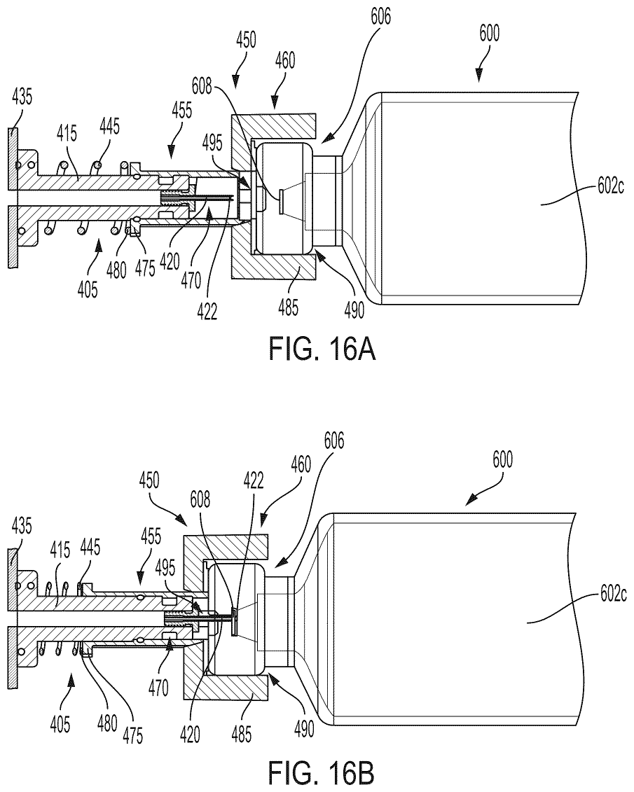

The biasing member 445 can be any suitable member for biasing the needle shield 450 relative to the needle support 415. In some examples, the biasing member 445 can be configured to receive the needle support 415. The biasing member 445 can be a spring such as, for example, a metal spring (e.g., a stainless steel spring) or a spring formed from any other suitable material for deforming to store potential energy that can be used to selectively bias the needle shield 450 as described below. The biasing member 445 can be any other suitable biasing member such as, for example, an elastic or flexible member (e.g., an elastomeric or rubber member) or any other suitable material for biasing the needle shield 450. The biasing member 445 can compress and expand relative to the needle support 415 and the septum piercing needle 420 based on a position of the needle shield 450 and the drug container 600 for example as described below and referring to FIGS. 16A and 16B.

Returning to FIG. 15, the needle shield 450 can be any suitable member for receiving a portion of the drug container 600 and selectively shielding the tip 422 of the septum piercing needle 420. The needle shield 450 can be disposed within the housing 440. For example, the needle shield 450 may not extend out of (e.g., outside of) the housing 440. The needle shield 450 can move based on biasing from the biasing member 445 and a position of the drug container 600 for example as described below and referring to FIGS. 17A and 17B. Returning to FIG. 15, the needle shield 450 can be formed from material similar to the needle support 415, the connecting member 425, and/or the housing 440 for example as described above.

The needle shield 450 can have any suitable dimensions and configuration for selectively covering and exposing the tip 422 of the septum piercing needle 420 and receiving a portion of the drug container 600. For example as illustrated in FIG. 15, the needle shield 450 can comprise a needle receiving portion 455 and a container receiving portion 460. The needle receiving portion 455 and the container receiving portion 460 can be integrally formed or can be separate portions that are attached using any suitable technique (e.g., bonding, welding such as ultrasonic welding, adhesives, and/or mechanical attachment such as via fasteners).

The needle receiving portion 455 can include a needle housing 465 that can form a needle cavity 470. The needle cavity 470 can selectively contain part or all of the septum piercing needle 420 for example as described further below. The needle housing 465 can be configured and dimensioned to be movably received by the needle support 415. In some examples, the needle support 415 can include one or more recesses or slots for receiving one or more portions of the needle housing 465 (or the needle housing 465 can include one or more recesses or slots for receiving one or more portions of the needle support 415) to guide a movement of the needle housing 465 along the needle support 415. The needle housing 465 can include a biasing member portion 475 (e.g., a protrusion or a lip) that can abut, engage, and/or be attached to an end portion 480 of the biasing member 445. The biasing member 445 can thereby be compressed via contact of the biasing member portion 475 against end portion 480. Biasing member 445 can bias needle shield 450 via contact of the end portion 480 against biasing member portion 475.

The container receiving portion 460 can be configured and dimensioned to receive a cap 606 of the drug container 600. The cap 606 can implement and be generally similar to the cap 206. The cap 606 can include a septum 608 that can implement and be generally similar to the septum 208. The container receiving portion 460 can include one or more receiving protrusions 485 that can form a container recess 490 that can receive (e.g., surround or enclose) some or substantially all of the cap 606. In some examples, the one or more receiving protrusions 485 (e.g., and/or the needle housing 465 of the needle receiving portion 455) can have a curved or annular shape.

Needle shield 450 can include a needle aperture 495 disposed between the container receiving portion 460 and the needle receiving portion 455. The needle aperture 495 can form a passage connecting the needle cavity 470 and the container recess 490. The tip 422 of the septum piercing needle 420 can pass through the needle aperture 495 based on a position of the drug container 600 and/or biasing of the needle shield 450 by the biasing member 445 for example as described further below.

Turning to FIGS. 16A and 16B, the needle shield 450, the biasing member 445, and the drug container 600 can move between a pre-pierced position (illustrated in FIG. 16A) and a pierced position (illustrated in FIG. 16B) relative to the needle assembly 405 (e.g., including the septum piercing needle 420) attached to the housing portion 435 of the housing 440. The drug container 600 can be moved from the pre-pierced position (illustrated in FIG. 16A) to the pierced position (illustrated in FIG. 16B) based on operation of a plunger (that can implement the plunger 112) as described further below.

As illustrated in FIG. 16A, in the pre-pierced position, the biasing member 445 can be uncompressed (e.g., storing substantially no potential energy). Although the biasing member portion 475 of the needle shield 450 can be in contact with the end portion 480 of the biasing member 445, the drug container 600 has not been moved by a plunger 500 (e.g., that can implement the plunger 112, as described below) so as to compress the biasing member 445 as described further below. The septum piercing needle 420 can be disposed entirely within the needle cavity 470 of the needle housing 465. The cap 606 can be received in the container recess 490.

As described further below, the plunger 500 can operate to move the drug container 600 from the pre-pierced position illustrated in FIG. 16A to the pierced position illustrated in FIG. 16B. As illustrated in FIG. 16B, in the pierced position, the biasing member 445 can be compressed (e.g., to store potential energy). Based on an operation of the plunger 500 as described further below, the drug container 600 can be moved toward the needle assembly 405. The cap 606 can contact and move the needle shield 450 toward the needle assembly 405 from the pre-pierced position illustrated in FIG. 16A toward the pierced position illustrated in FIG. 16B. As the needle shield 450 is moved toward the needle assembly 405, the needle receiving portion 455 can move along the needle support 415 toward housing portion 435, compressing the biasing member 445, based on the biasing member portion 475 of the needle receiving portion 455 biasing the end portion 480 of the biasing member 445. The tip 422 of the septum piercing needle 420 can pass through the needle aperture 495 as the needle shield 450 moves, until the septum 608 is pierced by the tip 422 of the septum piercing needle 420 as the cap 606 of the drug container 600 disposed in the container recess 490 of the container receiving portion 460 (of the needle shield 450) moves toward the needle assembly 405 (e.g., and into the pierced position illustrated in FIG. 16B). As described above, the needle assembly 405 including the septum piercing needle 420 can remain stationary, based on being attached to the housing portion 435 and the housing 440, as the needle shield 450 and the drug container 600 are moved from the pre-pierced position illustrated in FIG. 16A to the pierced position illustrated in FIG. 16B.

In the pierced position as illustrated in FIG. 16B, portions of the septum piercing needle 420 can be disposed in the needle cavity 470, the needle aperture 495, and the container recess 490 of the needle shield 450 so that the tip 422 pierces the septum 608 of the cap 606 disposed in container recess 490. Biasing member 445 can be compressed. Based on the septum piercing needle 420 including the hollow cavity that can be fluidly connected to the cavity 430 as described above, a fluid connection can be formed from a cavity 602c (that can implement cavity 202c) of the drug container 600 to the nozzle 101 via the septum piercing assembly 400.

Turning to FIGS. 17A and 17B, container receiving portion 460 of the needle shield 450 can be disposed in an opening 505 (that can implement opening 106) of the housing 440. The needle shield 450 can be moved between a shielded position as illustrated in FIG. 17A and an exposed position as illustrated in FIG. 17B. The shielded position of the needle shield 450 illustrated in FIG. 17A can correspond to the pre-pierced position of the needle shield 450 illustrated in FIG. 16A. The exposed position of the needle shield 450 illustrated in FIG. 17B can correspond to the pierced position of the needle shield 450 illustrated in FIG. 16B.

In the shielded position of the needle shield 450 illustrated in FIG. 17A, the tip 422 of the septum piercing needle 420 can be shielded (e.g., covered) by the needle shield 450. In the shielded position, the tip 422 may not be disposed in or pass through the needle aperture 495. The septum piercing needle 420 can be substantially entirely (e.g., entirely) disposed in the needle cavity 470 as illustrated in FIG. 16A when the needle shield 450 is in the shielded position illustrated in FIG. 17A. In some examples, a user (e.g., a finger of a user) of the drug delivery system 100 cannot come into contact with the tip 422 when the needle shield 450 is in the shielded position illustrated in FIG. 17A.

In the exposed position of the needle shield 450 illustrated in FIG. 17B, the tip 422 of the septum piercing needle 420 can extend through the needle shield 450. In the exposed position, the tip 422 can be disposed in and/or pass through the needle aperture 495. Portions of the septum piercing needle 420 can be disposed in the needle cavity 470, the needle aperture 495, and the container recess 490 as illustrated in FIGS. 16B and 17B when the needle shield 450 is in the exposed position of FIG. 17B (e.g., for clarity for showing the needle shield 450 and the septum piercing needle 420, the drug container 600 is not shown in FIG. 17B in opening 505). In some examples, the drug container 600 having the cap 606 disposed in the container recess 490 can have its septum 608 pierced by the tip 422 extending through the needle aperture 495 as illustrated in the pierced position of FIG. 16B and the exposed position of FIG. 17B (e.g., when the drug container 600 is disposed in the opening 505 when the needle shield 450 is in the exposed position).

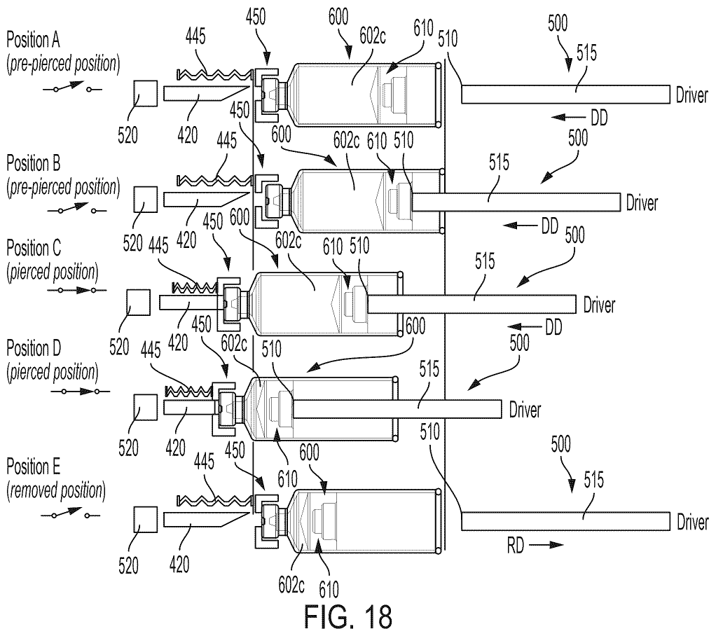

Turning to FIG. 18, an operation of the drug delivery system 100 including the septum piercing assembly 400 will now be described. As schematically illustrated in FIG. 18, the plunger 500 can move the drug container 600 relative to the septum piercing needle 420 of the needle assembly 405. The drug container 600 can include a seal 610 that can implement and be generally similar to seal 204. The plunger 500 can be any suitable plunger for moving the drug container 600 such as described above. For example, the plunger 500 can be generally similar to the plunger 302 described above. The plunger 500 can comprise a plunger end 510 and a flexible plunger rod 515. The flexible plunger rod 515 can be any suitable flexible plunger rod such as the examples described herein. For example, the flexible plunger rod 515 can be generally similar to the flexible plunger rod 306 described above. A driver that can for example be similar to the exemplary drivers described herein (e.g., the driver 114 or the driver 304) can drive the plunger 500.

At Position A as illustrated in FIG. 18, the plunger 500 can be in a pre-engaged position in which the plunger end 510 may not be in contact with the seal 610. The needle shield 450 can be in the shielded position (FIG. 17A). The drug container 600 can be placed in the opening 505 in the pre-pierced position (FIG. 16A).

The driver can operate to move the plunger 500 in a driving direction DD from the pre-engaged position at Position A to an engaged position at Position B. At Position B as illustrated in FIG. 18, the plunger 500 can be in the engaged position in which the plunger end 510 can be in contact with the seal 610. The needle shield 450 can be in the shielded position (FIG. 17A). The drug container 600 can be in the pre-pierced position (FIG. 16A).

The driver can operate to move the plunger 500 in the driving direction DD from the engaged position at Position B to an initial injection (dispensing) position at Position C. At Position C as illustrated in FIG. 18, the plunger end 510 can be in contact with the seal 610. The needle shield 450 can be in the exposed position (FIG. 17B). The drug container 600 can be in the pierced position (FIG. 16B).

In some examples, based on the drug disposed in cavity 602c being a substantially incompressible liquid, movement of the plunger 500 from position B (toward Position C) can move the drug container 600 against the needle shield 450, thereby moving the drug container 600 and the needle shield 450 toward needle assembly 405 and compressing the biasing member 445. For example, the pushing force applied by the plunger 500 against the seal 610 and the drug container 600 can be greater than a spring force of the biasing member 445 so that the biasing member 445 compresses. For example, the pushing force may be similar to the exemplary disclosed driving forces described above regarding actuator 111. For example as described above referring back to FIGS. 16A and 16B, the drug container 600 can be moved from the pre-pierced position of FIG. 16A to the pierced position of FIG. 16B in which the tip 422 of septum piercing needle 420 pierces the septum 608 of the drug container 600. The needle shield 450 can also be moved from the shielded position of FIG. 17A to the exposed position of FIG. 17B as the plunger 500 moves the drug container 600 from Position B to Position C. Accordingly at Position C, the plunger 500 can be at the initial injection (dispensing) position, the drug container 600 can be at the pierced position of FIG. 16B, and the needle shield 450 can be at the exposed position of FIG. 17B.

The driver can operate to move the plunger 500 in the driving direction DD from the initial injection (dispensing) position at Position C to a final injection (dispensing) position at Position D. At Position D as illustrated in FIG. 18, the plunger end 510 may be in contact with the seal 610. The needle shield 450 can be in the exposed position (FIG. 17B). The drug container 600 can be in the pierced position (FIG. 16B).

As the plunger 500 moves from Position C to Position D, the plunger end 510 can move the seal 610 within the cavity 602c to deliver the drug contained in the cavity 602c via the septum piercing needle 420. Accordingly (referring to Positions B, C, and D), during part of the travel of the plunger 500, the plunger 500 can push the drug container 600 to pierce the septum 608 against the tip 422 of the septum piercing needle 420 (movement of the plunger 500 from Position B to Position C), and during another part, the plunger 500 can push the seal 610 of the drug container 600 (movement of the plunger 500 from Position C to Position D).

The drug delivery system 100 can include a sensor 520 that can sense when the septum 608 is pierced at Position C so that a delivery of the drug from the drug container 600 can be controlled as the plunger 500 moves from Position C to Position D. The sensor 520 can measure the instant of time (e.g., the precise instant) when the septum 608 is pierced by the tip 422 of the septum piercing needle 420 and when the drug contained in the drug container 600 starts to be delivered. The sensor 520 can be any suitable displacement sensor such as, for example, a position sensor (e.g., linear position sensor) or a laser sensor. In some examples, the sensor 520 can measure a displacement of the needle shield 450. For example, an end stop of the needle shield 450 can occur at a time (e.g., exactly at a time) of fluid flow of the drug 20, which can be in time with (e.g., synchronized with) a limit switch actuation. Also for example, the sensor 520 can comprise a needle sensor such as a microsensor tip or any other suitable sensor for measuring when the septum 608 is pierced. In some examples, the sensor 520 can be integrated into the septum piercing needle. The sensor 520 can communicate with and be controlled by a controller such as, for example, the control circuitry 128 described above.

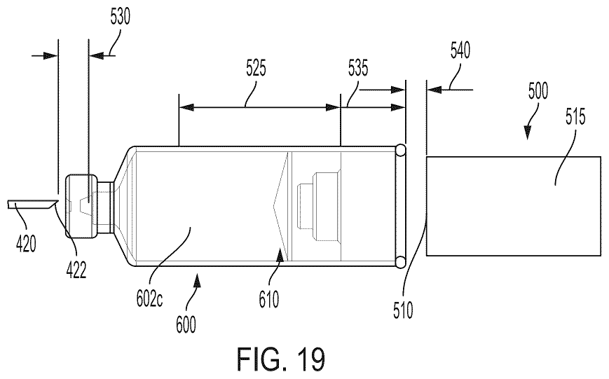

The controller (e.g., the controller circuitry 128) can determine when the plunger 500 moves the drug container to Position C and the septum 608 is pierced based on data or signals provided from the sensor 520, at which instant the plunger 500 has completed moving the entire drug container 600 and begins to move the seal 610 within the cavity 602c to deliver the drug via the septum piercing needle 420. (Delivery of the drug between Positions C and D is also schematically depicted in FIG. 18 based on the schematic switch that is shown as open at Positions A, B, and D also being shown as closed at Positions C and D to represent drug delivery.) Starting from the time and/or position that the septum 608 is pierced, the controller (e.g., the controller circuitry 128) can determine how much of the drug has been delivered based on the movement of the plunger 500. Accordingly, the amount of drug delivered can be controlled accurately (e.g., dose accuracy is provided) as further described below referring to FIG. 19. In some situations (e.g., weight-based dosing based on a weight of a patient), an entire container of drug may not be delivered (e.g., a portion of the drug container 600 less than the entire drug contained in the drug container can be delivered for a child). In some examples, this control can allow for the drug containers 600 to be of uniform size while being used to deliver different doses as suitable (e.g., the same-sized drug container 600 can be used to deliver a drug to an adult or to a child). This can reduce manufacturing costs by allowing the manufacturer to provide the same-sized drug container 600 to a wide variety of users (e.g., users of different age, weight, and other characteristics affecting dose amount). Also, as described above, the movement of the plunger 500 in the driving direction DD can provide a single mechanism that provides for both piercing of the septum 608 and delivery of the drug from the drug container 600.

The distance that the seal 610 is moved by the plunger 500 within the cavity 602c from Position C to Position D can determine the amount of drug that is delivered from the drug container 600. Referring to FIG. 19, a seal movement distance 525 depicts a distance that the seal 610 moves within the cavity 602c from Position C to Position D. The seal movement distance 525 can correspond to a plunger displacement that provides a corresponding amount of drug to be delivered (e.g., based on dimensions of the cavity 602c such as radius or width dimensions, which in combination with the seal movement distance 525 can correspond to a desired volume to be delivered). The seal movement distance 525 can be any suitable distance for drug delivery such as, for example, between about 10 mm and about 60 mm (e.g., about 45 mm). In some examples, an amount of drug delivered based on the plunger 500 moving the seal 610 from Position C to Position D can be between about 5 ml and about 30 ml. Dose accuracy can thereby be provided based on providing a drug delivery amount based on the seal movement distance 525. In some examples, the position of the seal 610 at Position D can provide a gap within the cavity 602c between the seal 610 and an end of the cavity 602c (e.g., the plunger 500 may not push the seal 610 into a front end of the drug container 600).

In some examples and as illustrated in FIG. 19, any suitable travel distance 530 can be provided for movement between Position B and Position C. For example, the travel distance 530 can be between about 5 mm and about 7 mm (e.g., about 6 mm). An initial engagement distance 535 can be any suitable distance between an end of the drug container 600 and an initial position of the seal 610 within the cavity 602c that is initially contacted by the plunger end 510 (e.g., at Position B). For example, the initial engagement distance 535 can be between about 1 mm and about 40 mm. An initial clearance distance 540 can be any suitable distance between the plunger end 510 and the end of the drug container 600 (e.g., at Position A) for providing initial clearance for an insertion of the drug container 600 into the opening 505. For example, the initial clearance distance 540 can be about 1 mm or about 2 mm, or can be greater than about 2 mm (e.g., based on dimensions of the housing 440).

Turning back to FIG. 18, the driver can operate to move the plunger 500 in a reverse direction RD, which can be opposite to the driving direction DD, from the final injection (dispensing) position at Position D to the post-engaged position at Position E. The plunger 500 can be moved in the reverse direction RD so that the plunger end 510 is removed from the cavity 602c as the plunger 500 moves from Position D to Position E. Accordingly, the driver can move the plunger 500 in both the driving direction DD and the reverse direction RD.

As the plunger 500 initially begins to move from Position D to Position E, the plunger end 510 can remain in contact with the seal 610. In some examples, the plunger end 510 can remain attached to the seal 610 (e.g., by any suitable technique such as mechanical interlock (e.g., snap fit), suction, or adhesion) as the plunger 500 moves in the reverse direction RD, which can urge or pull both the seal 610 and the drug container 600 in the reverse direction RD away from the needle assembly 405 (e.g., in embodiments in which a biasing member 445 may not be provided). In some examples, the plunger end 510 can remain attached to the seal 610 (e.g., which can be an elastomer seal, which can provide a relatively low release force) based on mechanical interlock such as snap fit. In other examples, as the plunger 500 moves in the reverse direction RD, biasing member 445 can bias needle shield 450 and the drug container 600 in the reverse direction RD so that the plunger end 510 remains in contact with the seal 610 as the plunger 500 moves in the reverse direction RD. For example, the biasing of biasing member 445 as it releases its stored potential energy (e.g., stored from moving from Position B to Position C) can bias the needle shield 450 to keep the drug container 600 pushed against the plunger 500 as the plunger 500 withdraws in the reverse direction RD. When the needle shield 450 reaches the shielded position (e.g., as illustrated in FIG. 17B), the biasing member 445 can stop biasing because it can be partially uncompressed with some or most stored potential energy having been released. In some examples, an end stop (e.g., of the housing 440) can control the position of the needle shield 450 in the shielded position. For example, the end stop can leave some spring force (e.g., about 5N in some embodiments) in the biasing member 445 to ensure that the septum piercing needle 420 fully retracts from the needle friction to the septum 608. The plunger 500 can continue to be moved in the reverse direction RD until the post-engaged position at Position E is reached. At Position E, the seal 610 can remain in the same position as Position D. For example, the seal 610 may be held in place due to a vacuum and, as such, remain in the same position in Position D and Position E. The plunger end 510 can disengage from the seal 610 as the plunger 500 moves from Position D to Position E.

At Position E as illustrated in FIG. 18, the plunger 500 can be in a post-engaged position in which the plunger end 510 may not be in contact with the seal 610 (e.g., similar to as in the pre-engaged position of Position A). The needle shield 450 can be in the shielded position (FIG. 17A). The drug container 600 can be in the post-pierced position, which can be similar to the pre-pierced position illustrated in FIG. 16A. The drug container 600 can then be removed from the opening 505 and replaced as desired during subsequent use of the drug delivery system 100.

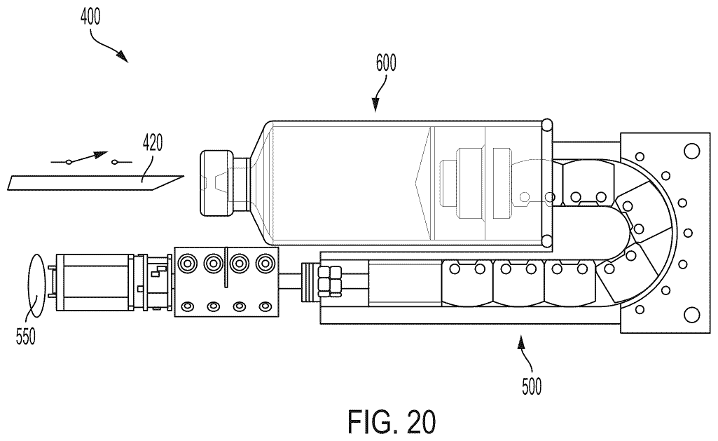

In some examples and referring to FIG. 20, a position control sensor 550 can be used for position control of the septum piercing assembly 400. The position control sensor 550 can be any suitable device for measuring a speed and/or a position of a rotational device such as, for example, rotational components of the driver and/or the plunger 500 (e.g., similar to the rotational components associated with the driver 304 and/or the plunger 302 for example as described above). The position control sensor 550 can be for example a rotary encoder such as a motor rotary shaft encoder. In some examples, the position control sensor 550 can be a magnetic rotary encoder or an optical rotary encoder. The position control sensor 550 can sense position and/or speed of rotational components of the driver and/or the plunger 500 as the plunger 500 is moved (e.g., as described above referring back to FIG. 18), which can be used to measure and control a position of the plunger 500 (e.g., regarding determining the seal movement distance 525). In some examples, an initial position of the plunger 500 can be determined using a position end switch (e.g., that can communicate with and/or be integrated into the control circuitry 128). Also in some examples, displacement of the drug container 600 and/or the plunger 500 can be controlled by any suitable pre-loading mechanism that can be integrated into the housing 440 (e.g., a spring-loaded assembly that can pre-load components of the plunger 500 such as chain components to remove slack).

In some examples, the drug delivery system 100 can comprise a curved track (e.g., similar to track 110), the plunger 500 having the flexible plunger rod 515 and the plunger end 510, the septum piercing needle 420, and the driver (e.g., similar to the driver 114 and/or the driver 304). The driver can be configured to cause the plunger 500 to translate along the curved track such that the flexible plunger rod 515 bends during translation along the curved track and the plunger 500 can be adapted to cause the drug container 600 to translate from a pre-pierced position at which the septum 608 of the drug container 600 is not pierced by the septum piercing needle 420 to a pierced position at which the septum 608 of the drug container 600 is pierced by the septum piercing needle 420. The plunger end 510 can be configured to engage the seal 610 of the drug container 600 to translate the drug container 600 from the pre-pierced position to the pierced position. When the drug container 600 is in the pierced position, movement of the flexible plunger rod 515 can cause the seal 610 of the drug container 600 to move within the drug container 600 to deliver the drug. The biasing assembly 410 can be configured to cause the drug container 600 to move from the pierced position to the removed position in which the septum piercing needle 420 is removed from the drug container 600. The sensor 520 can be configured to sense when the drug container 600 is moved to the pierced position. The controller (e.g., the controller circuitry 128) can control delivery of the drug based on the sensor 520 sensing when the drug container 600 is moved to the pierced position in which the delivery of the drug begins. When the drug container 600 is in the pierced position, the controller (e.g., the controller circuitry 128) can control the driver to cause the plunger 500 to translate along the curved track to move the plunger end 510 the seal movement distance 525. An amount of the drug delivered via the septum piercing needle 420 can be based on the seal movement distance 525.

In some examples, the drug delivery system 100 can comprise a curved track (e.g., similar to track 110), the plunger 500 having the flexible plunger rod 515 and the plunger end 510, the septum piercing needle 420 spaced from the plunger end 510, the septum piercing needle 420 configured to pierce the septum 608 of the drug container 600, and the driver (e.g., similar to the driver 114 and/or the driver 304). The driver can be configured to cause the plunger 500 to translate along the curved track along the driving direction DD such that the flexible plunger rod 515 bends along the curved track to drive the seal 610 of the drug container 600 to expel a liquid drug from the drug container 600, and to translate along the reverse direction RD, being opposite the driving direction DD, away from the seal 610 after expelling the liquid drug from the drug container 600 is complete. When the plunger 500 is translated along the reverse direction RD, the drug container 600 can move away from the septum piercing needle 420 to unpierce the septum 608. The biasing assembly 410 can be configured to translate the drug container 600 in the reverse direction RD so as to cause the septum piercing needle 420 to unpierce the septum 608. The biasing assembly 410 can comprise the needle shield 450 configured to house the tip 422 of the septum piercing needle 420 in the removed position when the drug container 600 is translated in the reverse direction RD. The biasing assembly 410 can include the biasing member 445 configured to translate the drug container 600 in the reverse direction RD when the plunger 500 is translated along the reverse direction RD. The plunger 500 including the plunger end 510 can be configured to pull the drug container 600 in the reverse direction RD. When the plunger 500 is translated along the reverse direction RD, the plunger 500 can be configured to move the drug container 600 away from the septum piercing needle 420 to unpierce the septum 608. The plunger 500 can be adapted to cause the drug container 600 to translate in the driving direction DD from the pre-pierced position at which the septum 608 of the drug container 600 is not pierced by the septum piercing needle 420 to the pierced position at which the septum 608 is pierced by the septum piercing needle 420. The flexible plunger rod 515 can be translatable between the disengaged position in which the plunger end 510 does not engage the drug container 600 and the engaged position in which the plunger end 510 engages the drug container 600. The driving direction DD and the reverse direction RD can be relative to the housing 440 that supports the septum piercing needle 420 in a stationary position. At least one roller or bearing disposed along the curved track can be configured to guide the flexible plunger rod 515 as the flexible plunger rod 515 translates along the curved track. A reinforcement structure can be configured to resist opposing forces applied by the plunger 500 at a curve defined by the curved track at a first end portion of the drug delivery system 100, and by the drug container 600 or the driver at a second end portion of the drug delivery system 100.

In some examples, the drug delivery system 100 can comprise a curved track (e.g., similar to track 110), the plunger 500 having the flexible plunger rod 515 and the plunger end 510, and the driver (e.g., similar to the driver 114 and/or the driver 304). The driver can be configured to cause the plunger 500 to translate along the curved track along the driving direction DD such that the flexible plunger rod 515 bends along the curved track to drive the seal 610 of the drug container 600 to expel the liquid drug from the drug container 600. The septum piercing needle 420 can be spaced from the plunger end 510, the septum piercing needle 420 configured to pierce the septum 608 of the drug container 600. The needle shield 450 can be configured to be moved between the shielding position, in which the needle shield 450 extends beyond the tip 422 of the septum piercing needle 420, and the exposed position, in which the tip 422 of the septum piercing needle 420 is exposed to allow the septum piercing needle 420 to pierce the septum 608 of the drug container 600. The needle shield 450 can be configured to move from the exposed position to the shielding position after the septum piercing needle 420 is removed from the septum 608 of the drug container 600. The biasing member 445 can bias the needle shield 450 from the exposed position to the shielding position when the plunger 500 is translated along the curved track along the reverse direction RD, opposite the driving direction DD, away from the seal 610. The needle shield 450 can move between the exposed position and the shielding position relative to the housing 440 that can support the septum piercing needle 420 in a stationary position.

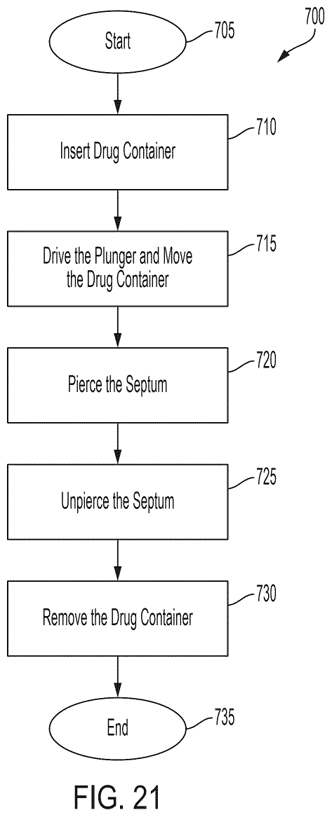

In some examples and referring to FIG. 21, an exemplary disclosed method (e.g., process 700) of using the drug delivery system 100 can comprise the following steps. Process 700 can begin at step 705. At step 710, process 700 can include inserting the drug container 600 into the opening 505 of the housing 440, the housing 440 including the septum-piercing needle 420. At step 715, process 700 can include driving the plunger 500 having the flexible plunger rod 515 and the plunger end 510 along a curved track, and moving the drug container 600 in the driving direction DD toward the septum-piercing needle 420 by pushing the drug container 600 with the plunger end 510. At step 720, process 700 can include piercing the septum 608 of the drug container 600 with the septum-piercing needle 420 based on driving the plunger 500 in the driving direction DD. At step 725, process 700 can include unpiercing the septum 608 of the drug container 600 with the septum-piercing needle 420 based on driving the plunger 500 in the reverse direction RD that is opposite to the driving direction DD. At step 730, process 700 can include removing the drug container 600 from the opening 505 of the housing 440. At step 735, process 700 can end.

In some examples regarding process 700, the driving direction DD and the reverse direction RD can be relative to the housing 440 that supports the septum piercing needle 420 in a stationary position. The needle shield 450 can be moved between the shielding position, in which the needle shield 450 extends beyond the tip 422 of the septum piercing needle 420, and the exposed position, in which the tip 422 of the septum piercing needle 420 is exposed to allow the septum piercing needle 420 to pierce the septum 608 of the drug container 600. The needle shield 450 can be configured to move from the exposed position to the shielding position after the septum piercing needle 420 is removed from the septum 608 of the drug container 600. The needle shield 450 can be biased from the exposed position to the shielding position using the biasing member 445 when the plunger 500 is translated along the curved track along the reverse direction RD.

It should be noted that the illustrations and descriptions of the examples and embodiments shown in the figures are for exemplary purposes only, and should not be construed limiting the disclosure. One skilled in the art will appreciate that the present disclosure contemplates various embodiments. Additionally, it should be understood that the concepts described above with the above-described examples and embodiments may be employed alone or in combination with any of the other examples and embodiments described above. It should further be appreciated that the various alternative examples and embodiments described above with respect to one illustrated embodiment can apply to all examples and embodiments as described herein, unless otherwise indicated.

Unless explicitly stated otherwise, each numerical value and range should be interpreted as being approximate as if the word “about,” “approximately,” or “substantially” preceded the value or range. The terms “about,” “approximately,” and “substantially” can be understood as describing a range that is within 20 percent, 15 percent, 10 percent, or 5 percent of a specified value unless otherwise stated.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

It will be understood that reference herein to “a” or “one” to describe a feature such as a component or step does not foreclose additional features or multiples of the feature. For instance, reference to a device having or defining “one” of a feature does not preclude the device from having or defining more than one of the feature, as long as the device has or defines at least one of the feature. Similarly, reference herein to “one of” a plurality of features does not foreclose the invention from including two or more, up to all, of the features. For instance, reference to a device having or defining “one of a protrusion and a recess” does not foreclose the device from having both the protrusion and the recess.

Aspects of the disclosure forming part of the description:

### Drug Delivery System Actuator

1. A drug delivery system, comprising:
at least one curved track;
   a plunger having a flexible plunger rod;
   a driver configured to cause the plunger to translate along the at least one curved track such that the flexible plunger rod bends as it translates along the at least one curved track into a drug container to drive a liquid drug from the drug container into a patient; and
   at least one roller or bearing configured to guide the flexible plunger rod as the flexible plunger rod translates along the at least one curved track.
2. The drug delivery system of aspect 1, wherein the flexible plunger rod supports the least one roller or bearing such that the least one roller or bearing rides along the at least one curved track with the flexible plunger rod.
3. The drug delivery system of aspect 1, wherein the track supports the least one roller or bearing such that plunger rod rides along the at least one roller or bearing.
4. The drug delivery system of any one of aspects 1 to 3, wherein the flexible plunger rod has a first outboard side and a second outboard side that are opposite one another, and the least one roller or bearing can comprise one or more rollers or bearings disposed on the first outboard side of the flexible plunger rod.
5. The drug delivery system of aspect 4, wherein:
   the at least one roller or bearing can comprise one or more rollers or bearings disposed on the second outboard side of the flexible plunger rod;
   the at least one track can comprises a pair of tracks that are opposite one another; and
   the one or more rollers or bearings of the first outboard side ride along a first one of the tracks, and the one or more rollers or bearings of the second outboard side ride along a second one of the tracks.
6. The drug delivery system of aspect 4, wherein the flexible plunger rod has a first outboard side and a second outboard side that are opposite one another, and the least one roller or bearing is disposed between the first outboard side and the second outboard side.
7. The drug delivery system of any one of aspects 1 to 6, wherein the flexible plunger rod comprises a plurality of links that are pivotably connected to one another.
8. The drug delivery system of aspect 6, wherein each of the at least one roller or bearing is supported by one link of the plurality of links.
9. The drug delivery system of any one of aspects 1 to 6, wherein the flexible plunger rod comprises a flexible material that is capable of bending as the plunger rod translates along the at least one curved track.
10. The drug delivery system of any one of aspects 1 to 9, comprising:
   a threaded rod that is configured to engage internal threads of the plunger; and
   a motor that is configured to cause the threaded rod to rotate to cause the plunger to translate along the at least one track.
11. The drug delivery system of any one of aspect 1 to 10, comprising reinforcement structure that is configured to resist opposing forces applied by the plunger at a curve defined by the at least one curved track at a first end of the drug delivery system, and by the drug container and/or driver at a second end of the drug delivery system, opposite the first end.
12. The drug delivery system of aspect 11, wherein the reinforcement structure comprises a rigid plate.
13. The drug delivery system of any one of aspects 11 and 12, wherein the reinforcement structure defines the at least one track.
14. The drug delivery system of any one of aspects 11 to 13, wherein the reinforcement structure has a first end that resists outward movement of the at least one curved track at a first end of the drug delivery system along a select direction, and a second end that resists outward movement of the drug container and/or driver at a second end of the drug delivery system along a direction opposite the select direction.
15. The drug delivery system of aspect 13, wherein the at least one track is defined by an opening or recess that extends into or through the reinforcement structure, the opening or recess configured to receive the at least one roller or bearing therein.
16. The drug delivery system of any one of aspects 1 to 15, comprising the drug container, wherein the drug container is a cartridge comprising a container body and a seal that forms a seal with an interior surface of the container body, and the plunger is configured to engage the seal to drive the liquid drug from the container.
17. The drug delivery system of any one of aspects 1 to 16, wherein the drug delivery system is configured such that a force needed to translate a seal within the drug container with the flexible plunger rod is no greater than 30% more than a force needed to translate the seal within the container with a straight plunger rod.
18. The drug delivery system of any one of aspects 1 to 17, wherein the drug delivery system is capable of driving the plunger seal within the container with a force of at least 50N.
19. The drug delivery system of any one of aspects 1 to 18, wherein the drug delivery system comprises:
   a needle or cannula; and
   an insertion mechanism configured to insert a needle or cannula of the drug delivery system into a patient.
20. A method of delivering a drug to a patient with a drug delivery system, the method comprising:
   inserting a needle or cannula of the drug delivery system into the patient; and
   causing a flexible plunger rod of the drug delivery system to translate along at least one curved track of the drug delivery system such that the flexible plunger rod bends as it translates along the at least one curved track into a drug container of the drug delivery system to drive a liquid drug from the drug container into the patient, wherein at least one roller or bearing of the of the drug delivery system guides the flexible plunger rod as the flexible plunger rod translates along the at least one curved track.
21. The method of aspect 20, wherein the flexible plunger rod supports the least one roller or bearing, and the causing step comprises causing the least one roller or bearing to ride along the at least one curved track with the flexible plunger rod.
22. The method of aspect 20, wherein the track supports the least one roller or bearing, and the causing step comprises causing the plunger rod to ride along the at least one roller or bearing.
23. The method of any one of aspects 20 to 22, wherein the drug delivery system comprises a threaded rod that is configured to engage internal threads of the plunger, and the method comprises causing a driver to cause the threaded rod to rotate to cause the plunger to translate along the at least one track.
24. A drug delivery system, comprising:
   a curved track;
   a plunger having a flexible plunger rod;
   a driver configured to cause the plunger to translate along the curved track such that the flexible plunger rod bends along the curved track to drive a plunger seal of a drug container to expel a liquid drug from the drug container,
   wherein the drug delivery system is configured such that a force needed for the driver to translate the plunger seal within the container with the flexible plunger rod is no greater than 30% more than a force needed to translate the plunger seal within the container with a straight plunger rod.
25. The drug delivery system of aspect 24, wherein the drug delivery system is configured such that the force needed for the driver to translate the plunger seal within the container with the flexible plunger rod is no greater than 25% more than the force needed to translate the plunger seal within the container with a straight plunger rod.
26. The drug delivery system of aspect 24, wherein the drug delivery system is configured such that the force needed for the driver to translate the plunger seal within the container with the flexible plunger rod is no greater than 20% more than the force needed to translate the plunger seal within the container with a straight plunger rod.
27. The drug delivery system of aspect 24, wherein the drug delivery system is configured such that the force needed for the driver to translate the plunger seal within the container with the flexible plunger rod is no greater than 15% more than the force needed to translate the plunger seal within the container with a straight plunger rod.
28. The drug delivery system of any one of aspects 24 to 27, comprising:
   a threaded rod that is configured to engage internal threads of the plunger; and
   a motor that is configured to cause the threaded rod to rotate to cause the plunger to translate along the at least one track.
29. The drug delivery system of any one of aspect 24 to 28, comprising reinforcement structure that is configured to resist opposing forces applied by the plunger at a curve defined by the at least one curved track at a first end of the drug delivery system, and by the drug container and/or driver at a second end of the drug delivery system, opposite the first end.
30. The drug delivery system of aspect 29, wherein the reinforcement structure comprises a rigid plate.
31. The drug delivery system of any one of aspects 29 to 30, wherein the reinforcement structure has a first end that resists outward movement of the at least one curved track at a first end of the drug delivery system along a select direction, and a second end that resists outward movement of the drug container and/or driver at a second end of the drug delivery system along a direction opposite the select direction.
32. The drug delivery system of any one of aspects 24 to 31, comprising the drug container, wherein the drug container is a cartridge comprising a container body and a seal that forms a seal with an interior surface of the container body, and the plunger is configured to engage the seal to drive the liquid drug from the container.
33. The drug delivery system of any one of aspects 24 to 32, wherein the drug delivery system is capable of driving the plunger seal within the container with a force of at least 50N.
34. The drug delivery system of any one of aspects 24 to 33, wherein the drug delivery system comprises:
   a needle or cannula; and
   an insertion mechanism configured to insert a needle or cannula of the drug delivery system into a patient.
35. A drug delivery system, comprising:
   a curved track;
   a plunger having a flexible plunger rod;
   a driver configured to cause the plunger to translate along the curved track such that the flexible plunger rod bends along the curved track to drive a plunger seal of a drug container to expel a liquid drug from the drug container,
   wherein the drug delivery system is capable of driving the plunger seal within the container with a force of at least 50N.
36. The drug delivery system of aspect 35, wherein the drug delivery system is capable of driving the plunger seal within the container with a force of at least 150N.
37. The drug delivery system of aspect 35, wherein the drug delivery system is capable of driving the plunger seal within the container with a force of at least 200N.
38. The drug delivery system of aspect 35, wherein the drug delivery system is capable of driving the plunger seal within the container with a force of at least 250N.
39. The drug delivery system of aspect 35, wherein the drug delivery system is capable of driving the plunger seal within the container with a force of at least 300N.
40. The drug delivery system of any one of aspects 35 to 39, comprising:
   a threaded rod that is configured to engage internal threads of the plunger; and
   a motor that is configured to cause the threaded rod to rotate to cause the plunger to translate along the at least one track. 41. The drug delivery system of any one of aspect 35 to 40, comprising reinforcement structure that is configured to resist opposing forces applied by the plunger at a curve defined by the at least one curved track at a first end of the drug delivery system, and by the drug container and/or driver at a second end of the drug delivery system, opposite the first end.
42. The drug delivery system of aspect 41, wherein the reinforcement structure comprises a rigid plate.
43. The drug delivery system of any one of aspects 41 to 42, wherein the reinforcement structure has a first end that resists outward movement of the at least one curved track at a first end of the drug delivery system along a select direction, and a second end that resists outward movement of the drug container and/or driver at a second end of the drug delivery system along a direction opposite the select direction.
44. The drug delivery system of any one of aspects 35 to 43, comprising the drug container, wherein the drug container is a cartridge comprising a container body and a seal that forms a seal with an interior surface of the container body, and the plunger is configured to engage the seal to drive the liquid drug from the container.
45. The drug delivery system of any one of aspects 40 to 44, wherein the drug delivery system comprises:
   a needle or cannula; and
   an insertion mechanism configured to insert a needle or cannula of the drug delivery system into a patient.
46. A method of delivering a drug to a patient with a drug delivery system, the method comprising:
   inserting a needle or cannula of the drug delivery system into the patient; and
   causing a flexible plunger rod of the drug delivery system to translate along at least one curved track of the drug delivery system such that the flexible plunger rod bends as it translates along the at least one curved track into a drug container of the drug delivery system to drive a liquid drug from the drug container with a force of at least 50N.
47. The method of aspect 46, wherein the drug delivery system is capable of driving the plunger seal within the container with a force of at least 150N.
48. The method of aspect 46, wherein the drug delivery system is capable of driving the plunger seal within the container with a force of at least 200N.
49. The method of aspect 46, wherein the drug delivery system is capable of driving the plunger seal within the container with a force of at least 250N.
50. The method of aspect 46, wherein the drug delivery system is capable of driving the plunger seal within the container with a force of at least 300N.
51. The method of any one of aspects 46 to 50, wherein the drug delivery system comprises a threaded rod that is configured to engage internal threads of the plunger, and the method comprises causing a driver to cause the threaded rod to rotate to cause the plunger to translate along the at least one track.

### Septum Piercing Assembly

1. A drug delivery system, comprising:
   a curved track;
   a plunger having a flexible plunger rod and a plunger end;
   a septum piercing needle; and
   a driver configured to cause the plunger to translate along the curved track such that the flexible plunger rod bends during translation along the curved track and the plunger is adapted to cause a drug container to translate from a pre-pierced position at which a septum of the drug container is not pierced by the septum piercing needle to a pierced position at which the septum of the drug container is pierced by the septum piercing needle.
2. The drug delivery system of aspect 1, wherein the plunger end is configured to engage a seal of the drug container to translate the drug container from the pre-pierced position to the pierced position.
3. The drug delivery system of any one of aspects 1 to 2, wherein when the drug container is in the pierced position, movement of the flexible plunger rod causes a seal of the drug container to move within the drug container to deliver the drug.
4. The drug delivery system of any one of aspects 1 to 3, further comprising a biasing assembly configured to cause the drug container to move from the pierced position to a removed position in which the septum piercing needle is removed from the drug container.
5. The drug delivery system of any one of aspects 1 to 4, further comprising a sensor configured to sense when the drug container is moved to the pierced position.
6. The drug delivery system of aspect 5, further comprising a controller that controls delivery of the drug based on the sensor sensing when the drug container is moved to the pierced position in which the delivery of the drug begins.
7. The drug delivery system of any one of aspects 1 to 5, further comprising a controller;
   wherein when the drug container is in the pierced position, the controller controls the driver to cause the plunger to translate along the curved track to move the plunger end a predetermined distance.
8. The drug delivery system of aspect 7, wherein an amount of the drug delivered via the septum piercing needle is based on the predetermined distance.
9. A drug delivery system, comprising:
   a curved track;
   a plunger having a flexible plunger rod and a plunger end;
   a septum piercing needle spaced from the plunger end, the septum piercing needle configured to pierce a septum of the drug container; and
   a driver configured to cause the plunger to translate along the curved track along a driving direction such that the flexible plunger rod bends along the curved track to drive a seal of the drug container to expel a liquid drug from the drug container, and to translate along a reverse direction, being opposite the driving direction, away from the seal after expelling the liquid drug from the drug container is complete,
   wherein when the plunger is translated along the reverse direction, the drug container moves away from the septum piercing needle to unpierce the septum.
10. The drug delivery system of aspect 9, further comprising a biasing assembly configured to translate the drug container in the reverse direction so as to cause the septum piercing needle to unpierce the septum.
11. The drug delivery system of aspect 10, wherein the biasing assembly comprises a needle shield configured to house a tip of the septum piercing needle in a removed position when the drug container is translated in the reverse direction.
12. The drug delivery system of any one of aspects 10 to 11, wherein the biasing assembly includes a biasing member configured to translate the drug container in the reverse direction when the plunger is translated along the reverse direction.
13. The drug delivery system of any one of aspects 9 to 12, wherein the plunger including the plunger end is configured to pull the drug container in the reverse direction,
   wherein when the plunger is translated along the reverse direction, the plunger is configured to move the drug container away from the septum piercing needle to unpierce the septum.
14. The drug delivery system of any one of aspects 9 to 13, wherein the plunger is adapted to cause the drug container to translate in the driving direction from a pre-pierced position at which the septum of the drug container is not pierced by the septum piercing needle to a pierced position at which the septum is pierced by the septum piercing needle.
15. The drug delivery system of any one of aspects 9 to 14, wherein the flexible plunger rod is translatable between a disengaged position in which the plunger end does not engage the drug container and an engaged position in which the plunger end engages the drug container.
16. The drug delivery system of any one of aspects 9 to 15, wherein the driving direction and the reverse direction are relative to a housing that supports the septum piercing needle in a stationary position.
17. The drug delivery system of any one of aspects 9 to 16, further comprising at least one roller or bearing disposed along the curved track and configured to guide the flexible plunger rod as the flexible plunger rod translates along the curved track.
18. The drug delivery system of any one of aspects 9 to 17, further comprising a reinforcement structure that is configured to resist opposing forces applied by the plunger at a curve defined by the curved track at a first end portion of the drug delivery system, and by the drug container or the driver at a second end portion of the drug delivery system.
19. A drug delivery system, comprising:
   a curved track;
   a plunger having a flexible plunger rod and a plunger end;
   a driver configured to cause the plunger to translate along the curved track along a driving direction such that the flexible plunger rod bends along the curved track to drive a seal of the drug container to expel the liquid drug from the drug container;
   a septum piercing needle spaced from the plunger end, the septum piercing needle configured to pierce a septum of the drug container; and
   a needle shield configured to be moved between a shielding position, in which the needle shield extends beyond a tip of the septum piercing needle, and an exposed position, in which the tip of the septum piercing needle is exposed to allow the septum piercing needle to pierce the septum of the drug container.
20. The drug delivery system of aspect 19, wherein the needle shield is configured to move from the exposed position to the shielding position after the septum piercing needle is removed from the septum of the drug container.
21. The drug delivery system of aspect 20, further comprising a biasing member that biases the needle shield from the exposed position to the shielding position when the plunger is translated along the curved track along a reverse direction, opposite the driving direction, away from the seal.
22. The drug delivery system of aspect 19, wherein the needle shield moves between the exposed position and the shielding position relative to a housing that supports the septum piercing needle in a stationary position.
23. A method, comprising:
   driving a plunger having a flexible plunger rod and a plunger end along a curved track,
      the plunger and the curved track being situated inside a housing,
      the plunger being arranged to interact with a drug container situated inside the housing,
      the housing having a septum-piercing needle;
   moving the drug container in a driving direction toward the septum-piercing needle by pushing the drug container with the plunger end;
   piercing a septum of the drug container with the septum-piercing needle based on driving the plunger in the driving direction; and
   unpiercing the septum of the drug container with the septum-piercing needle based on driving the plunger in a reverse direction that is opposite to the driving direction.
24. The method of aspect 23, wherein the driving direction and the reverse direction are relative to the housing that supports the septum piercing needle in a stationary position.
25. The method of any one of aspects 23 and 24, further comprising:
   moving a needle shield between a shielding position, in which the needle shield extends beyond a tip of the septum piercing needle, and an exposed position, in which the tip of the septum piercing needle is exposed to allow the septum piercing needle to pierce the septum of the drug container.
26. The method of any one of aspects 23 to 25, wherein the needle shield is configured to move from the exposed position to the shielding position after the septum piercing needle is removed from the septum of the drug container.
27. The method of aspect 26, further comprising:
   biasing the needle shield from the exposed position to the shielding position when the plunger is translated along the curved track along the reverse direction.
28. The method of any one of aspects 23 to 27, further comprising:
   providing an opening in the housing to receive the drug container in the housing and to remove the drug container from the housing.

1. A drug delivery system, comprising:
a curved track;
a plunger having a flexible plunger rod and a plunger end;
a septum piercing needle; and
a driver configured to cause the plunger to translate along the curved track such that the flexible plunger rod bends during translation along the curved track and the plunger is adapted to cause a drug container to translate from a pre-pierced position at which a septum of the drug container is not pierced by the septum piercing needle to a pierced position at which the septum of the drug container is pierced by the septum piercing needle.

2. The drug delivery system of claim 1, wherein the plunger end is configured to engage a seal of the drug container to translate the drug container from the pre-pierced position to the pierced position.

3. The drug delivery system of claim 1, wherein when the drug container is in the pierced position, movement of the flexible plunger rod causes a seal of the drug container to move within the drug container to deliver the drug.

4. The drug delivery system of claim 1, further comprising a biasing assembly configured to cause the drug container to move from the pierced position to a removed position in which the septum piercing needle is removed from the drug container.

5. The drug delivery system of claim 1, further comprising a sensor configured to sense when the drug container is moved to the pierced position.

6. The drug delivery system of claim 5, further comprising a controller that controls delivery of the drug based on the sensor sensing when the drug container is moved to the pierced position in which the delivery of the drug begins.

7. The drug delivery system of claim 1, further comprising a controller;
wherein when the drug container is in the pierced position, the controller controls the driver to cause the plunger to translate along the curved track to move the plunger end a predetermined distance.

8. The drug delivery system of claim 7, wherein an amount of the drug delivered via the septum piercing needle is based on the predetermined distance.

9. A drug delivery system, comprising:
a curved track;
a plunger having a flexible plunger rod and a plunger end;
a septum piercing needle spaced from the plunger end, the septum piercing needle configured to pierce a septum of the drug container; and
a driver configured to cause the plunger to translate along the curved track along a driving direction such that the flexible plunger rod bends along the curved track to drive a seal of the drug container to expel a liquid drug from the drug container, and to translate along a reverse direction, being opposite the driving direction, away from the seal after expelling the liquid drug from the drug container is complete,
wherein when the plunger is translated along the reverse direction, the drug container moves away from the septum piercing needle to unpierce the septum.

10. The drug delivery system of claim 9, further comprising a biasing assembly configured to translate the drug container in the reverse direction so as to cause the septum piercing needle to unpierce the septum.

11. The drug delivery system of claim 10, wherein the biasing assembly comprises a needle shield configured to house a tip of the septum piercing needle in a removed position when the drug container is translated in the reverse direction.

12. The drug delivery system of claim 10, wherein the biasing assembly includes a biasing member configured to translate the drug container in the reverse direction when the plunger is translated along the reverse direction.

13. The drug delivery system of claim 9, wherein the plunger including the plunger end is configured to pull the drug container in the reverse direction,
wherein when the plunger is translated along the reverse direction, the plunger is configured to move the drug container away from the septum piercing needle to unpierce the septum.

14. The drug delivery system of claim 9, wherein the plunger is adapted to cause the drug container to translate in the driving direction from a pre-pierced position at which the septum of the drug container is not pierced by the septum piercing needle to a pierced position at which the septum is pierced by the septum piercing needle.

15. The drug delivery system of claim 9, wherein the flexible plunger rod is translatable between a disengaged position in which the plunger end does not engage the drug container and an engaged position in which the plunger end engages the drug container.

16-18. (canceled)

19. A drug delivery system, comprising:
a curved track;
a plunger having a flexible plunger rod and a plunger end;
a driver configured to cause the plunger to translate along the curved track along a driving direction such that the flexible plunger rod bends along the curved track to drive a seal of the drug container to expel the liquid drug from the drug container;
a septum piercing needle spaced from the plunger end, the septum piercing needle configured to pierce a septum of the drug container; and
a needle shield configured to be moved between a shielding position, in which the needle shield extends beyond a tip of the septum piercing needle, and an exposed position, in which the tip of the septum piercing needle is exposed to allow the septum piercing needle to pierce the septum of the drug container.

20. The drug delivery system of claim 19, wherein the needle shield is configured to move from the exposed position to the shielding position after the septum piercing needle is removed from the septum of the drug container.

21. The drug delivery system of claim 20, further comprising a biasing member that biases the needle shield from the exposed position to the shielding position when the plunger is translated along the curved track along a reverse direction, opposite the driving direction, away from the seal.

22. The drug delivery system of claim 19, wherein the needle shield moves between the exposed position and the shielding position relative to a housing that supports the septum piercing needle in a stationary position.

23. A method, comprising:
driving a plunger having a flexible plunger rod and a plunger end along a curved track,
the plunger and the curved track being situated inside a housing,
the plunger being arranged to interact with a drug container situated inside the housing,
the housing having a septum-piercing needle;
moving the drug container in a driving direction toward the septum-piercing needle by pushing the drug container with the plunger end;
piercing a septum of the drug container with the septum-piercing needle based on driving the plunger in the driving direction; and
unpiercing the septum of the drug container with the septum-piercing needle based on driving the plunger in a reverse direction that is opposite to the driving direction.

24-28. (canceled)
